(19) United States
(12) Patent Application Publication
(10) Pub. No.: US 2021/0347709 A1
(43) Pub. Date: Nov. 11, 2021

(54) RADIOFLUORINATED GPC3-BINDING PEPTIDES FOR PET IMAGING OF HEPATOCELLULAR CARCINOMA

(71) Applicant: University of Southern California, Los Angeles, CA (US)

(72) Inventors: Kai Chen, Los Angeles, CA (US); Peter S. CONTI, Los Angeles, CA (US)

(73) Assignee: University of Southern California, Los Angeles, CA (US)

(21) Appl. No.: 17/245,938

(22) Filed: Apr. 30, 2021

Related U.S. Application Data
(60) Provisional application No. 63/018,576, filed on May 1, 2020.

(51) Int. Cl. C07B 59/00; A61K 51/10; G01N 33/534

(52) U.S. Cl. CPC C07B 59/002; G01N 33/534; A61K 51/1093

(57) ABSTRACT

The invention provides a radiopharmaceutical compound or composition comprising a radiolabeled linear peptide that binds specifically to Glypican-3 (GPC3) expressed on a surface of a cell. Preferably, the linear peptide is conjugated to one or more ¹⁸F atoms.

### RELATED APPLICATIONS

This application claims priority under 35 U.S.C. § 119(e) to U.S. Provisional Patent Application No. 63/018,576, filed May 1, 2020, which is incorporated herein by reference.

### SEQUENCE LISTING

The instant application contains a Sequence Listing which has been submitted electronically in ASCII format and is hereby incorporated by reference in its entirety. Said ASCII copy, created on Apr. 26, 2021, is named 530_016US1_SL.txt and is 1,101 bytes in size.

### GOVERNMENT SUPPORT

This invention was made with government support under grant number P30 DK048522 awarded by the National Institutes of Health. The government has certain rights in the invention.

### BACKGROUND OF THE INVENTION

Liver cancer is a major health problem, which currently ranks the fourth leading cause of cancer death worldwide. Among all primary liver cancers, hepatocellular carcinoma (HCC) is the most common type, representing 75%-85% of all primary liver cancer cases. In the United States, HCC incidence and mortality rates have been increasing for decades. Median survival following diagnosis of HCC is approximately 6 to 20 months due to late diagnosis in its course and few effective treatment options. Although surgical resection or liver transplantation may sometimes be successful for the treatment of early-stage HCC, very limited treatment options are available for patients diagnosed at an advanced stage. Definitive diagnosis via non-invasive testing of HCC in clinic includes four-phase multi-detector computed tomography (CT) or dynamic contrast-enhanced magnetic resonance imaging (MRI). Further improvement in HCC patient management through imaging will be limited unless anatomical studies are augmented with an assessment of tumor biology and metabolism in vivo. A major factor contributed to this limitation is an inability to characterize tumor growth and metabolism, a matter of pathophysiology which cannot be evaluated by anatomic imaging techniques.

Targeted therapies for the management of patients with HCC continue to be researched. However, the treatment responses are still being assessed on the basis of tumor size measurement before and after therapy. As targeted therapies may not cause significant changes in the size of lesions at an early stage, assessment of response to such treatments may not be accurate using conventional size measurement. The development of positron emission tomography (PET)/x-ray computed tomography (CT) technology creates the opportunity to combine metabolic and anatomic imaging capabilities, capitalizing on the advantages each modality affords. A sizable body of evidence suggests that the basic pathophysiological processes of HCC may be evaluable in vivo using the physiologic imaging capabilities of PET. Therefore, a target-specific PET probe may provide the early detection of HCC and/or be used as a companion diagnostic for HCC therapy.

Glypicans (GPCs) are a family of heparan sulfate proteoglycans anchored to cell membrane. Among six identified GPCs in mammals, Glypican-3 (GPC3) is an oncofetal proteoglycan containing a 70 kDa core protein. There is no GPC3 expression in healthy liver, but GPC3 expression remains at high levels in HCCs. In addition, the research results showed that, during the invasive growth of liver cancer, GPC3 expresses at different levels, suggesting that GPC3 plays an important role on HCC development. Furthermore, HCC cell migration and invasion can be inhibited by GPC3 knockdown, indicating GPC3 may also critically involve in HCC metastasis and invasion.

Due to the important role of GPC3 in the HCC progression, various GPC3-targeted therapies have been developed, including antibodies, vaccines, immunotoxins, and genetic therapies. Companion diagnostics for antibody-based HCC therapies have been recently reported, where an anti-human GPC3 mAb (DFO-1G12) or αGPC3 IgG1 and the fragments were radiolabeled with ⁸⁹Zr for PET imaging of HepG2 tumors. Although ⁸⁹Zr-labeled GPC3 mAbs showed very good HCC targeting efficacy and specificity, the relatively large size of mAbs led to unfavorable in vivo pharmacokinetics (PK) and immunogenicity, which might limit their clinical applications. Accordingly, there is a need for a small, target specific probe to provide clinicians with vital diagnostic as well as treatment of HCC patients. The present invention satisfies these needs.

### SUMMARY OF THE INVENTION

Through screening of a peptide library using immunoprecipitation method, a tetrakaideca peptide (TP) having the sequence RLNVGGTYFLTTRQ (SEQ ID NO: 1) was identified as a specific ligand binding to GPC3. This ligand can block the binding of the FITC-labeled TP to GPC3-expressing cells, further indicating the specific binding of TP for GPC3. As compared to antibodies, peptides usually show less immunogenicity and toxicity, and the production cost of peptides is relatively lower. Thus, it has become our great interest of utilizing the TP as a platform to build up GPC3-targeted PET probes. Recently, we radiolabeled the TP with F-18 to form a PET probe for imaging GPC3 positive HCC tumors. However, a low tumor/liver ratio was observed due to high hepatobiliary excretion. In contrast, the new PET probes described herein reduce the background radioactivity in liver and thus increase the tumor-to-liver (T/L) ratio.

Accordingly, the disclosure therefore provides a pharmaceutical composition comprising a radiolabeled linear peptide that binds specifically to Glypican-3 (GPC3) expressed on a surface of a cell, and a pharmaceutically acceptable carrier. Preferably, the GPC3 is expressed on the surface of a hepatocarcinoma cell.

In certain embodiments of the disclosure, the radiolabeled linear peptide is conjugated to one or more ¹⁸F atoms. In certain preferred embodiment of the invention, the radiolabeled linear peptide is conjugated to Al[¹⁸F]F.

In some embodiments, the ¹⁸F labeled PET probes incorporate a linker into the TP (SEQ ID NO: 1).

In some embodiments, the probes incorporate a hydrophilic peptide linker (GGGRDN) (SEQ ID NO: 3) into the TP (SEQ ID NO: 1). The new PET probe having the sequence GGGRDNRLNVGGTYFLTTRQ (SEQ ID NO: 2) may reduce the background radioactivity in liver and thus increase the tumor-to-liver (T/L) ratio.

In certain embodiments of the disclosure, the radiolabeled linear peptide is at least 75%, at least 80% at least 85%, at least 90%, at least 95%, or at least 100% sequence identity to the amino acid sequence RLNVGGTYFLTTRQ (SEQ ID NO: 1) or GGGRDNRLNVGGTYFLTTRQ (SEQ ID NO: 2). In other preferred embodiments of the invention, the radiolabeled linear peptide is selected from a peptide disclosed in Scheme 1A and Scheme 1B.

The invention also provides for a radiopharmaceutical compound comprising a radiolabeled linear peptide comprising RLNVGGTYFLTTRQ (SEQ ID NO: 1), or a radiolabeled linear peptide having a structure disclosed in Scheme 1A or 1B. In certain embodiments of the invention, the linear peptide is conjugated to one or more ¹⁸F atoms. In certain preferred embodiment of the invention, the radiolabeled linear peptide is conjugated to Al[¹⁸F]F.

The invention also provides for a radiopharmaceutical compound comprising two or more linear peptides, a central joint moiety wherein each of the two or more linear peptides is connected to the central joint moiety via a linker, and a functionalized linker connected to the central joint moiety wherein the functionalized linker includes one or more radiolabeled moieties.

In preferred embodiments of the invention, the two or more linear peptides of the radiopharmaceutical compound comprise one or more of SEQ ID NO: 1 or SEQ ID NO: 2. And preferably, each of the linker and the functionalized linker is a hydrophilic moiety comprising one or more of a polyethylene glycol unit, a sugar, or a short peptide.

### BRIEF DESCRIPTION OF THE DRAWINGS

The following drawings form part of the specification and are included to further demonstrate certain embodiments or various aspects of the invention. In some instances, embodiments of the invention can be best understood by referring to the accompanying drawings in combination with the detailed description presented herein. The description and accompanying drawings may highlight a certain specific example, or a certain aspect of the invention. However, one skilled in the art will understand that portions of the example or aspect may be used in combination with other examples or aspects of the invention.

FIG. 1 illustrates Cell uptake and efflux assay. (a) Time dependent uptake of Al[¹⁸F]F-GP2076 (dot dash line) and Al[¹⁸F]F-GP2633 (red line) in GPC3-positive HepG2 cells, and Al[¹⁸F]F-GP2633 (dash dash line) in GPC3-negative McA-RH7777 cells (n=4/group, mean±SD). (b) Time dependent efflux of Al[¹⁸F]F-GP2076 (dot dash line) and Al[18F]F-GP2633 (solid line) in GPC3-positive HepG2 cells, and Al[¹⁸F]F-GP2633 (dash dash line) in GPC3-negative McA-RH7777 cells (n=4/group, mean±SD).

FIG. 2 illustrates metabolic stability of (a) Al[¹⁸F]F-GP2076 or (b) Al[¹⁸F]F-GP2633 in HepG2 tumor, blood, liver, kidneys, and urine at 1 h pi. The analytical HPLC profile of Al[¹⁸F]F-GP2076 or Al[¹⁸F]F-GP2633 is shown as a reference.

FIG. 3 illustrates microPET-CT study of subcutaneous HCC bearing nude mice after 1 h intravenous (i.v.) injection of Al[¹⁸F]F-GP2076 or Al[¹⁸F]F-GP2633. Tumors are indicated by grey arrows, and livers are indicated by white arrows. Representative decay-corrected whole-body coronal microPET-CT images of nude mice bearing GPC3-positive HepG2 tumor after 1 h i.v. injection of (a) Al[¹⁸F]F-GP2076 or (b) Al[¹⁸F]F-GP2633. (c) Representative decay-corrected whole-body coronal microPET-CT images of nude mice bearing GPC3-negative McA-RH7777 tumor after 1 h i.v. injection of Al[¹⁸F]F-GP2633.

FIG. 4 illustrates the biodistribution of Al[¹⁸F]F-GP2076 or Al[¹⁸F]F-GP2633 at 30, 60, and 120 min pi in major tissues and organs of subcutaneous HCC bearing nude mice (n=3/group; mean±SD). Left bar: Al[¹⁸F]F-GP2076 in GPC3-positive HepG2 tumor model; Middle bar: Al[¹⁸F]F-GP2633 in GPC3-positive HepG2 tumor model; Right bar: Al[¹⁸F]F-GP2633 in GPC3-negative McA-RH7777 tumor model. (a) Uptake in tumor (% ID/g). (b) Uptake in liver (% ID/g). (c) Uptake in kidneys (% ID/g). (d) Tumor-to-muscle (T/M) ratio. (e) Tumor-to-liver (T/L) ratio. (f) Tumor-to-kidneys (T/K) ratio. Statistical significance between two groups is shown (*P<0.05; **P<0.01; ***P<0.001; NS, non-significant).

FIG. 5 illustrates immunohistochemical (IHC) staining of (a) HepG2 tumor and (b) McA-RH7777 tumor for GPC3, and hematoxylin and eosin (H&E) staining of (c) HepG2 tumor and (d) McA-RH7777 tumor (Scale bar: 100 μm). The IHC staining confirmed that the HepG2 tumor is GPC3 positive, and the McA-RH7777 tumor is GPC3 negative.

FIG. 6 illustrates mass spectrometry characterization of chemical structures of the peptides shown in Scheme 1. (a) The mass spectra demonstrate that Al[¹⁹F]F-GP2076 was successfully formed by chelating Al¹⁹F to the NOTA of GP2076. (b) The mass spectra demonstrate that Al[¹⁹F]F-GP2633 was successfully formed by chelating Al¹⁹F to the NOTA of GP2633.

FIG. 7 illustrates the analytical HPLC UV profile of GP2076 (a1) or GP2633 (a2) at 214 nm. The analytical HPLC radioactivity profile of the crude product of Al[¹⁸F]F-GP2076 (b1) or Al[¹⁸F]F-GP2633 (b2). The HPLC radioactivity profile of Al[¹⁸F]F-GP2076 (c1) or Al[¹⁸F]F-GP2633 (c2) after purification.

FIG. 8 illustrates HepG2 cell viability after the incubation with GP2076 or GP2633 at the peptide concentrations of 120, 240, 360, 480, 600, 720, and 840 μg/mL for 24 h. HepG2 cells incubated with PBS were used as a control.

FIG. 9 illustrates cell uptake and internalization assay. (a) Time dependent cell uptake (solid line) and internalization (dotted line) of Al[¹⁸F]F-GP2076 in GPC3-positive HepG2 cells. (b) Time dependent cell uptake (solid line) and internalization (dotted line) of Al[¹⁸F]F-GP2633 in GPC3-positive HepG2 cells. (c) Time dependent cell uptake (solid line) and internalization (dotted line) of Al[¹⁸F]F-GP2633 in GPC3-negative McA-RH7777 cells. (n=4/group, mean±SD). FIG. 10 illustrates in vitro stability of Al[¹⁸F]F-GP2076 or Al[¹⁸F]F-GP2633 in PBS and mouse serum. The HPLC radioactivity profile of Al[¹⁸F]F-GP2076 (a1) or Al[¹⁸F]F-GP2633 (a2) as a reference. The HPLC radioactivity profile of Al[¹⁸F]F-GP2076 (b1) or Al[¹⁸F]F-GP2633 (b2) after the incubation of radiolabeled tracers in PBS (pH=7.4) at room temperature for 2 h. The HPLC radioactivity profile of Al[¹⁸F]F-GP2076 (c1) or Al[¹⁸F]F-GP2633 (c2) after the incubation of radiolabeled tracers in mouse serum at 37° C. for 2 h.

FIG. 11 illustrates MicroPET images of subcutaneous HCC bearing nude mice after intravenous (i.v.) injection of Al[¹⁸F]F-GP2076 or Al[¹⁸F]F-GP2633. Tumors are indicated by white circles. Left: Representative decay-corrected whole-body coronal microPET images of nude mice bearing GPC3-positive HepG2 tumor after the i.v. injection of Al[¹⁸F]F-GP2076 at 30, 60, and 120 min. Middle: Representative decay-corrected whole-body coronal microPET images of nude mice bearing GPC3-positive HepG2 tumor after the i.v. injection of Al[¹⁸F]F-GP2633 at 30, 60, and 120 min. Right: Representative decay-corrected whole-body coronal microPET images of nude mice bearing GPC3-negative McA-RH7777 tumor after the i.v. injection of Al[¹⁸F]F-GP2633 at 30, 60, and 120 min.

FIG. 12 illustrates a representative microPET images of continuous whole-body coronal slices of HepG2 tumor-bearing mice at 60 min after the i.v. injection of Al[¹⁸F]F-GP2076 (a) or Al[¹⁸F]F-GP2633 (b). Tumors are indicated by gray arrows, and livers are indicated by white arrows.

### DETAILED DESCRIPTION

### Definitions

The following definitions are included to provide a clear and consistent understanding of the specification and claims. As used herein, the recited terms have the following meanings. All other terms and phrases used in this specification have their ordinary meanings as one of skill in the art would understand. Such ordinary meanings may be obtained by reference to technical dictionaries, such as Hawley's Condensed Chemical Dictionary 14^{th} Edition, by R. J. Lewis, John Wiley & Sons, New York, N.Y., 2001.

References in the specification to “one embodiment”, “an embodiment”, etc., indicate that the embodiment described may include a particular aspect, feature, structure, moiety, or characteristic, but not every embodiment necessarily includes that aspect, feature, structure, moiety, or characteristic. Moreover, such phrases may, but do not necessarily, refer to the same embodiment referred to in other portions of the specification. Further, when a particular aspect, feature, structure, moiety, or characteristic is described in connection with an embodiment, it is within the knowledge of one skilled in the art to affect or connect such aspect, feature, structure, moiety, or characteristic with other embodiments, whether or not explicitly described.

The singular forms “a,” “an,” and “the” include plural reference unless the context clearly dictates otherwise. Thus, for example, a reference to “a compound” includes a plurality of such compounds, so that a compound X includes a plurality of compounds X. It is further noted that the claims may be drafted to exclude any optional element. As such, this statement is intended to serve as antecedent basis for the use of exclusive terminology, such as “solely,” “only,” and the like, in connection with any element described herein, and/or the recitation of claim elements or use of “negative” limitations.

The term “and/or” means any one of the items, any combination of the items, or all of the items with which this term is associated. The phrases “one or more” and “at least one” are readily understood by one of skill in the art, particularly when read in context of its usage. For example, the phrase can mean one, two, three, four, five, six, ten, 100, or any upper limit approximately 10, 100, or 1000 times higher than a recited lower limit. For example, one or more substituents on a phenyl ring refers to one to five, or one to four, for example if the phenyl ring is disubstituted.

As will be understood by the skilled artisan, all numbers, including those expressing quantities of ingredients, properties such as molecular weight, reaction conditions, and so forth, are approximations and are understood as being optionally modified in all instances by the term “about.” These values can vary depending upon the desired properties sought to be obtained by those skilled in the art utilizing the teachings of the descriptions herein. It is also understood that such values inherently contain variability necessarily resulting from the standard deviations found in their respective testing measurements. When values are expressed as approximations, by use of the antecedent “about,” it will be understood that the particular value without the modifier “about” also forms a further aspect.

The term “about” can refer to a variation of ±5%, ±10%, ±20%, or ±25% of the value specified. For example, “about 50” percent can in some embodiments carry a variation from 45 to 55 percent, or as otherwise defined by a particular claim. For integer ranges, the term “about” can include one or two integers greater than and/or less than a recited integer at each end of the range. Unless indicated otherwise herein, the term “about” is intended to include values, e.g., weight percentages, proximate to the recited range that are equivalent in terms of the functionality of the individual ingredient, composition, or embodiment. The term about can also modify the end-points of a recited range as discussed above in this paragraph.

As will be understood by one skilled in the art, for any and all purposes, particularly in terms of providing a written description, all ranges recited herein also encompass any and all possible sub-ranges and combinations of sub-ranges thereof, as well as the individual values making up the range, particularly integer values. It is therefore understood that each unit between two particular units are also disclosed. For example, if 10 to 15 is disclosed, then 11, 12, 13, and 14 are also disclosed, individually, and as part of a range. A recited range (e.g., weight percentages or carbon groups) includes each specific value, integer, decimal, or identity within the range. Any listed range can be easily recognized as sufficiently describing and enabling the same range being broken down into at least equal halves, thirds, quarters, fifths, or tenths. As a non-limiting example, each range discussed herein can be readily broken down into a lower third, middle third and upper third, etc. As will also be understood by one skilled in the art, all language such as “up to”, “at least”, “greater than”, “less than”, “more than”, “or more”, and the like, include the number recited and such terms refer to ranges that can be subsequently broken down into sub-ranges as discussed above. In the same manner, all ratios recited herein also include all sub-ratios falling within the broader ratio. Accordingly, specific values recited for radicals, substituents, and ranges, are for illustration only; they do not exclude other defined values or other values within defined ranges for radicals and substituents. It will be further understood that the endpoints of each of the ranges are significant both in relation to the other endpoint, and independently of the other endpoint.

One skilled in the art will also readily recognize that where members are grouped together in a common manner, such as in a Markush group, the invention encompasses not only the entire group listed as a whole, but each member of the group individually and all possible subgroups of the main group. Additionally, for all purposes, the invention encompasses not only the main group, but also the main group absent one or more of the group members. The invention therefore envisages the explicit exclusion of any one or more of members of a recited group. Accordingly, provisos may apply to any of the disclosed categories or embodiments whereby any one or more of the recited elements, species, or embodiments, may be excluded from such categories or embodiments, for example, for use in an explicit negative limitation.

The term “contacting” refers to the act of touching, making contact, or of bringing to immediate or close proximity, including at the cellular or molecular level, for example, to bring about a physiological reaction, a chemical reaction, or a physical change, e.g., in a solution, in a reaction mixture, in vitro, or in vivo.

An “effective amount” refers to an amount effective to treat a disease, disorder, and/or condition, or to bring about a recited effect. For example, an effective amount can be an amount effective to reduce the progression or severity of the condition or symptoms being treated. Determination of a therapeutically effective amount is well within the capacity of persons skilled in the art. The term “effective amount” is intended to include an amount of a compound described herein, or an amount of a combination of compounds described herein, e.g., that is effective to treat or prevent a disease or disorder, or to treat the symptoms of the disease or disorder, in a host. Thus, an “effective amount” generally means an amount that provides the desired effect. An appropriate “effective” amount in any individual case may be determined using techniques, such as a dose escalation study.

The terms “treating”, “treat” and “treatment” include (i) preventing a disease, pathologic or medical condition from occurring (e.g., prophylaxis); (ii) inhibiting the disease, pathologic or medical condition or arresting its development; (iii) relieving the disease, pathologic or medical condition; and/or (iv) diminishing symptoms associated with the disease, pathologic or medical condition. Thus, the terms “treat”, “treatment”, and “treating” can extend to prophylaxis and can include prevent, prevention, preventing, lowering, stopping or reversing the progression or severity of the condition or symptoms being treated. As such, the term “treatment” can include medical, therapeutic, and/or prophylactic administration, as appropriate.

The terms “inhibit”, “inhibiting”, and “inhibition” refer to the slowing, halting, or reversing the growth or progression of a disease, infection, condition, or group of cells. The inhibition can be greater than about 20%, 40%, 60%, 80%, 90%, 95%, or 99%, for example, compared to the growth or progression that occurs in the absence of the treatment or contacting.

As used herein, “sequence identity” or “identity” in the context of two nucleic acid or polypeptide sequences makes reference to a specified percentage of residues in the two sequences that are the same when aligned for maximum correspondence over a specified comparison window, as measured by sequence comparison algorithms or by visual inspection. When percentage of sequence identity is used in reference to proteins it is recognized that residue positions which are not identical often differ by conservative amino acid substitutions, where amino acid residues are substituted for other amino acid residues with similar chemical properties (e.g., charge or hydrophobicity) and therefore do not change the functional properties of the molecule. When sequences differ in conservative substitutions, the percent sequence identity may be adjusted upwards to correct for the conservative nature of the substitution. Sequences that differ by such conservative substitutions are said to have “sequence similarity” or “similarity.” Means for making this adjustment are well known to those of skill in the art. Typically this involves scoring a conservative substitution as a partial rather than a full mismatch, thereby increasing the percentage sequence identity. Thus, for example, where an identical amino acid is given a score of 1 and a non-conservative substitution is given a score of zero, a conservative substitution is given a score between zero and 1. The scoring of conservative substitutions is calculated, e.g., as implemented in the program PC/GENE (Intelligenetics, Mountain View, Calif.).

As used herein, “percentage of sequence identity” means the value determined by comparing two optimally aligned sequences over a comparison window, wherein the portion of the polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison, and multiplying the result by 100 to yield the percentage of sequence identity.

The term “substantial identity” in the context of a peptide indicates that a peptide comprises a sequence with at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, or 94%, or even 95%, 96%, 97%, 98% or 99%, sequence identity to the reference sequence over a specified comparison window. In certain embodiments, optimal alignment is conducted using the homology alignment algorithm of Needleman and Wunsch (Needleman and Wunsch, JMB, 48, 443 (1970)). An indication that two peptide sequences are substantially identical is that one peptide is immunologically reactive with antibodies raised against the second peptide. Thus, a peptide is substantially identical to a second peptide, for example, where the two peptides differ only by a conservative substitution. Thus, the invention also provides nucleic acid molecules and peptides that are substantially identical to the nucleic acid molecules and peptides presented herein.

For sequence comparison, typically one sequence acts as a reference sequence to which test sequences are compared. When using a sequence comparison algorithm, test and reference sequences are input into a computer, subsequence coordinates are designated if necessary, and sequence algorithm program parameters are designated. The sequence comparison algorithm then calculates the percent sequence identity for the test sequence(s) relative to the reference sequence, based on the designated program parameters.

Nucleic acid sequences cited herein are written in a 5′ to 3′ direction unless indicated otherwise. The term “nucleic acid” refers to either DNA or RNA or a modified form thereof comprising the purine or pyrimidine bases present in DNA (adenine “A”, cytosine “C”, guanine “G”, thymine “T”) or in RNA (adenine “A”, cytosine “C”, guanine “G”, uracil “U”). Interfering RNAs provided herein may comprise “T” bases, for example at 3′ ends, even though “T” bases do not naturally occur in RNA. In some cases, these bases may appear as “dT” to differentiate deoxyribonucleotides present in a chain of ribonucleotides.

The terms “polypeptide,” “peptide,” and “protein” are used interchangeably herein to refer to polymers of amino acids of any length. The polymer may be linear or branched, it may comprise modified amino acids, and it may be interrupted by non-amino acids. The terms also encompass an amino acid polymer that has been modified naturally or by intervention; for example, disulfide bond formation, glycosylation, lipidation, acetylation, phosphorylation, or any other manipulation or modification, such as fusion with another polypeptide and/or conjugation, e.g., with a labeling component. Also included within the definition are, for example, polypeptides containing one or more analogs of an amino acid (for example, unnatural amino acids, etc.), as well as other modifications known in the art.

Variants of the peptide sequences can be readily selected by one of skill in the art, based in part on known properties of the sequence. For example, a variant peptide can include amino acid substitutions (e.g., conservative amino acid substitutions) and/or deletions (e.g., small, single amino acid deletions, or deletions encompassing 2, 3, 4, 5, 10, 15, 20, or more contiguous amino acids). Thus, in certain embodiments, a variant of a native peptide sequence is one that differs from a naturally-occurring sequence by (i) one or more (e.g., 2, 3, 4, 5, 6, or more) conservative amino acid substitutions, (ii) deletion of 1 or more (e.g., 2, 3, 4, 5, 6, or more) amino acids, or (iii) a combination thereof. Deleted amino acids can be contiguous or non-contiguous. Conservative amino acid substitutions are those that take place within a family of amino acids that are related in their side chains and chemical properties. These include, e.g., (1) acidic amino acids: aspartate, glutamate; (2) basic amino acids: lysine, arginine, histidine; (3) nonpolar amino acids: alanine, valine, leucine, isoleucine, proline, phenylalanine, methionine, tryptophan; (4) uncharged polar amino acids: glycine, asparagine, glutamine, cysteine, serine, threonine, tyrosine; (5) aliphatic amino acids: glycine, alanine, valine, leucine, isoleucine, serine, threonine, with serine and threonine optionally grouped separately as aliphatic-hydroxyl; (6) aromatic amino acids: phenylalanine, tyrosine, tryptophan; (7) amide amino acids: asparagine, glutamine; and (9) sulfur-containing amino acids: cysteine and methionine. See, e.g., Biochemistry, 2nd ed., Ed. by L. Stryer, W H Freeman and Co.: 1981. Methods for confirming that variant peptides are suitable are conventional and routine.

### Embodiments of the Technology

This disclosure provides a binding probe of Formula I:

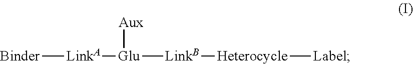

wherein

Label is Al[¹⁸F], ¹⁸F, Al[¹⁹F], ¹⁹F, or absent, wherein the bond to ¹⁸F or F is ionic or covalent;

Aux is absent or -Link^{C}-Binder;

Glu is absent or

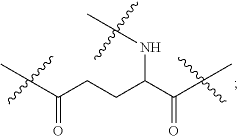 [SMILES: *NC(CCC(*)=O)C(*)=O]

Link^{A} is absent, a polyethylene glycol (PEG), or the short peptide GGG;

Link^{B} is

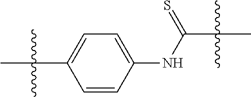 [SMILES: *C(=S)Nc1ccc(*)cc1]

a monosaccharide, PEG, or combination thereof;

Link^{C} is absent, a polyethylene glycol (PEG), or the short peptide GGG;
each Binder is independently a binding peptide (GP) or RGD;
wherein the binding peptide has a sequence identity of at least about 75% to the amino acid sequence -GGGRDNRLNVGGTYFLTTRQ (SEQ ID NO: 2) or -RLNVGGTYFLTTRQ (SEQ ID NO: 1);
wherein RGD is:

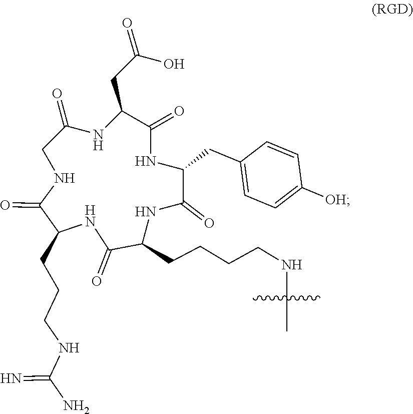 [SMILES: *NCCCC[C@@H]1NC(=O)[C@@H](Cc2ccc(O)cc2)NC(=O)[C@H](CC(=O)O)NC(=O)CNC(=O)[C@H](CCCNC(=N)N)NC1=O]

wherein the N-terminus or the binding peptide or the (CH₂)₄NH moiety of RGD is conjugated to Link^{B}, Link^{A}, or Glu (when Link^{A} and/or Link^{C} absent), and the conjugate is via a thioacyl or acyl bond; and

Heterocycle is:

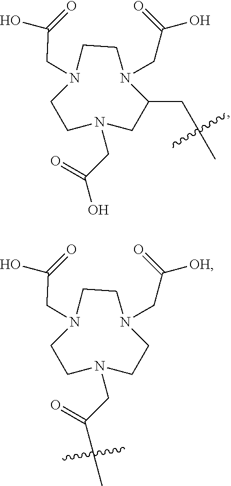

an imidazolyl, triazolyl, tetrazolyl, pyridyl, or combination thereof of any two of the heterocycles;
wherein Glu is not absent when Link^{A} and/or Link^{C} are not absent; and
each PEG independently has a molecular weight of about 20 kDa or less.

In some embodiments, Link^{A} is conjugated via one or two amide bonds. In some embodiments, the heterocycle and/or monosaccharide and/or PEG is conjugated by one or more amide bonds. In some embodiments, the sequence identity is at least about 85%.

In some embodiments, PEG is represented by Formula Ib or Ibi:

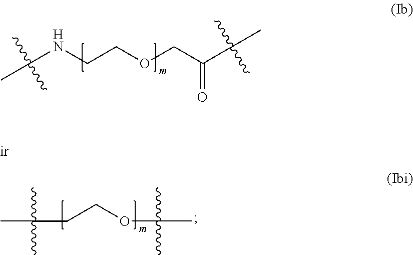

wherein m is 1-2000. In some embodiments, m is 1-200, 1-100, 1-50, 1-25, 1-10, 2-20, 2-10, or about 5. In some other embodiments, m is 1, 2, 3, 4, 5, 6, 7, 8, 9, or 10.

In some embodiments, the monosaccharide is represented by Formula Ic:

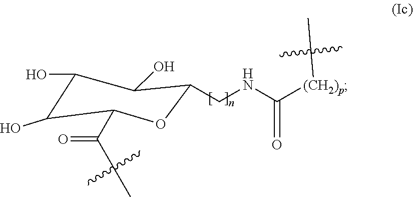

wherein n and p are each independently 0-2000.

In some embodiments, the triazolyl is represented by Formula Id or Ie:

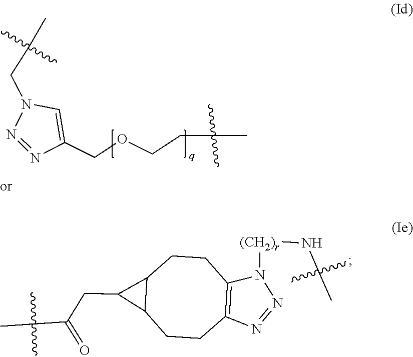

wherein q is 1-10; and r is 2-10.

In some embodiments, the pyridyl is represented by Formula If or Ifi:

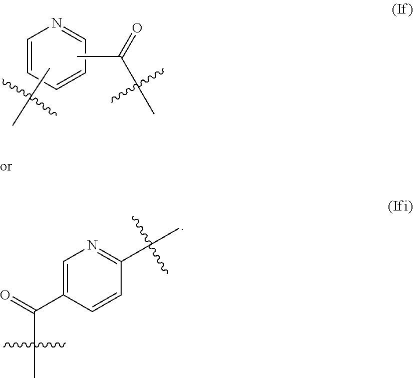

In some embodiments, the Heterocycle is represented by Formula Ig:

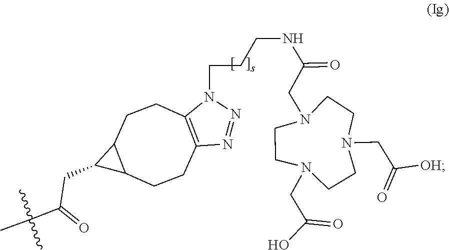

wherein s is 0-8.

In various embodiments, the Label is Al[¹⁸F] or ¹⁸F. In various embodiments, both Aux and Glu are not absent. In various embodiments, both Link^{A} and Link^{C} are not absent.

In various embodiments, the probe is represented by Formula II

Binder—Link^{B}—Heterocycle—Label (II);

wherein

Label is Al[¹⁸F] or ¹⁸F;

Binder is GP or RGD; and

Heterocycle is:

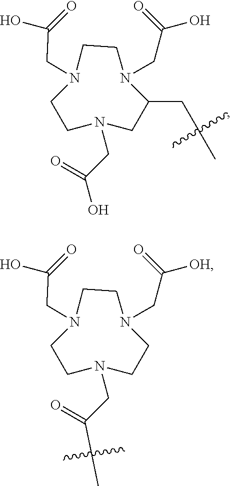

triazolyl, or pyridyl; wherein the sequence identity is at least about 95%.

In some embodiments, the probe is:

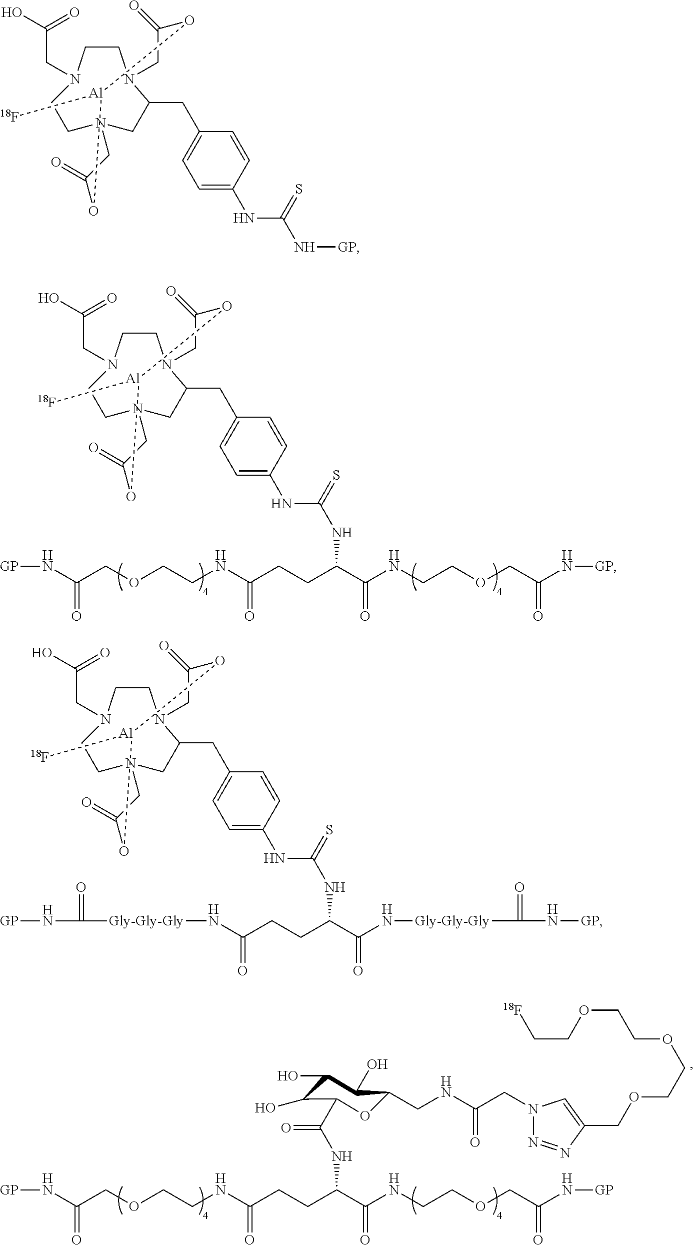

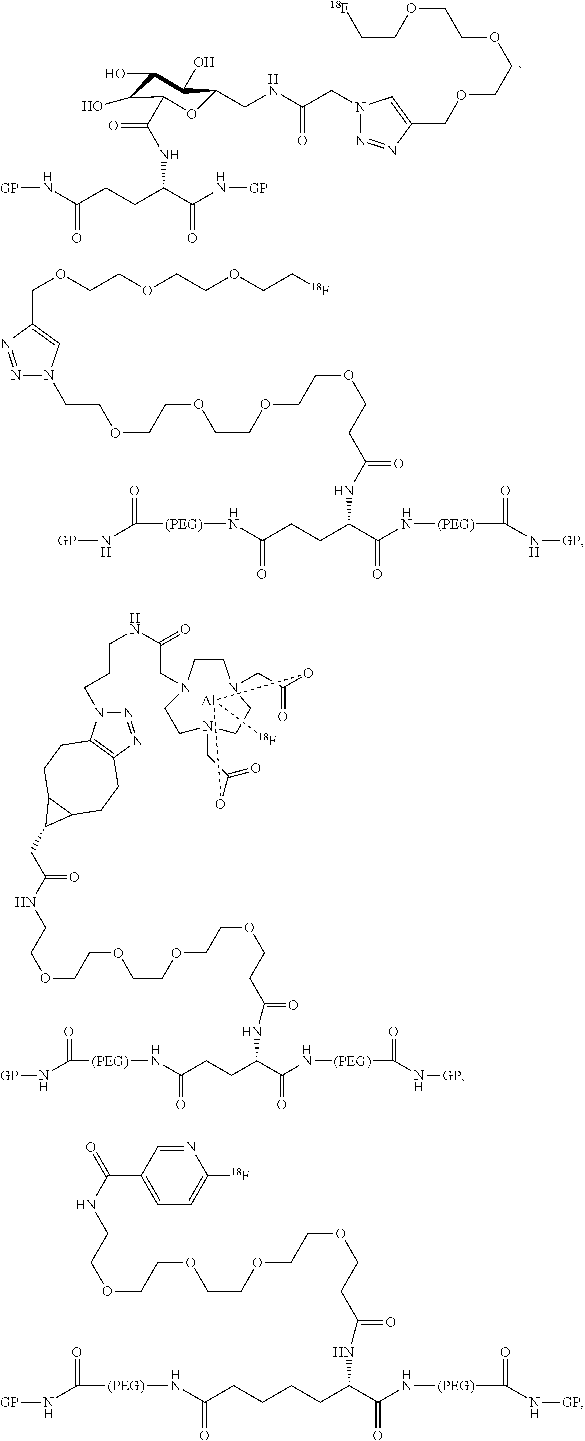

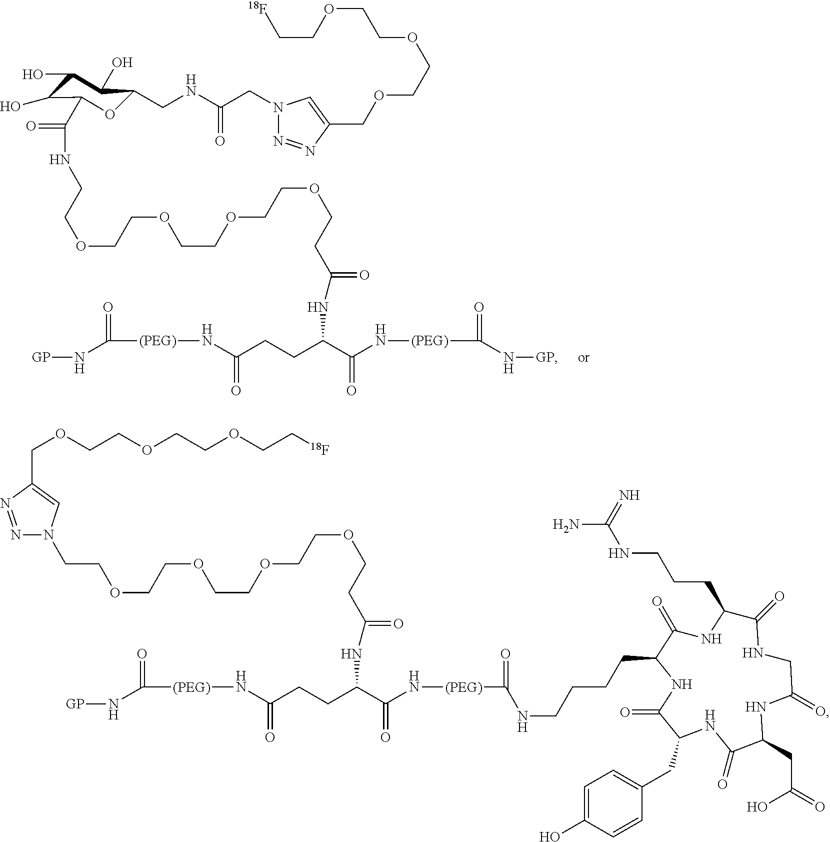

wherein PEG consists of 4 repeat units; and GP is -GGGRDNRLNVGGTYFLTTRQ (SEQ ID NO: 2) wherein the amine moiety of the amino acid G at the N-terminus of GP is represented as NH. In various embodiments, the sequence identity is about 100%.

This disclosure also provides a method for imaging a cancer comprising:
a) administering an effective amount of the binding probe disclosed herein; and b) imaging the presence or absence of the cancer in the subject.

In various other embodiments, the probe is Al[¹⁸F]F-GP2633 or Al[¹⁸F]F-GP2076:

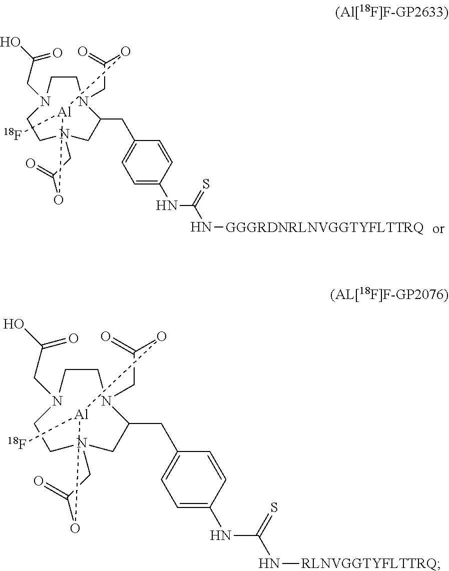

wherein the amine moiety of the amino acid G at the N-terminus of GGGRDNRLNVGGTYFLTTRQ (SEQ ID NO: 2) is represented as NH which forms the thiourea moiety in Al[¹⁸F]F-GP2633;
wherein the amine moiety of the amino acid R at the N-terminus of RLNVGGTYFLTTRQ (SEQ ID NO: 1) is represented as NH which forms the thiourea moiety in Al[¹⁸F]F-GP2026.

In some embodiments, the sequence identity is about 95%, about 99% or 100%. In some embodiments, the cancer is liver cancer or hepatocarcinoma.

Additionally, this disclosure provides a radiopharmaceutical composition comprising an ¹⁸F radiolabeled linear peptide that binds specifically to Glypican-3 (GPC3) expressed on a surface of a hepatocarcinoma cell; and a pharmaceutically acceptable carrier.

In some embodiments, the radiolabeled linear peptide is at least 75% identical to amino acid sequence RLNVGGTYFLTTRQ (SEQ ID NO: 1) or GGGRDNRLNVGGTYFLTTRQ (SEQ ID NO: 2). In some other embodiments, the radiolabeled linear peptide is at least 95% identical to amino acid sequence RLNVGGTYFLTTRQ (SEQ ID NO: 1) or GGGRDNRLNVGGTYFLTTRQ (SEQ ID NO: 2).

### Additional Aspects of the Technology

This disclosure provides a radiopharmaceutical composition comprising a radiolabeled linear peptide that binds specifically to Glypican-3 (GPC3) expressed on a surface of a cell; and a pharmaceutically acceptable carrier.

In some aspects, GPC3 is expressed on a hepatocarcinoma cell. In some aspects, the radiolabeled linear peptide is conjugated to one or more ¹⁸F atoms. In some aspects, the radiolabeled linear peptide is at least 75% identical to amino acid sequence RLNVGGTYFLTTRQ (SEQ ID NO: 1). In some aspects, the radiolabeled linear peptide is RLNVGGTYFLTTRQ (SEQ ID NO: 1).

In some aspects, the radiopharmaceutical composition further comprising a linker moiety disposed at an N-terminus of the radiolabeled linear peptide. In some aspects, the linker moiety is GGGRDN (SEQ ID NO: 3). In some aspects, the radiolabeled linear peptide is at least 75% identical to amino acid sequence GGGRDNRLNVGGTYFLTTRQ (SEQ ID NO: 2). In some aspects, the radiolabeled linear peptide is GGGRDNRLNVGGTYFLTTRQ (SEQ ID NO: 2). In some aspects, the radiolabeled linear peptide is selected from a peptide disclosed in Scheme 1a or Scheme 1b.

Furthermore, this disclosure provides a radiopharmaceutical compound comprising a radiolabeled linear peptide comprising RLNVGGTYFLTTRQ (SEQ ID NO: 1).

Additionally, this disclosure provides a radiopharmaceutical compound comprising a radiolabeled linear peptide having a structure disclosed in Scheme 1a or Scheme 1b.

This disclosure also provides a radiopharmaceutical compound comprising two or more linear peptides;
a central joint moiety wherein each of the two or more linear peptides is connected to the central joint moiety via a linker; and
a functionalized linker connected to the central joint moiety wherein the functionalized linker includes one or more radiolabeled moieties.

In some aspects, the two or more linear peptides comprise one or more of SEQ ID NO: 1 or SEQ ID NO: 2. In other aspects, each of the linker and the functionalized linker is a hydrophilic moiety comprising one or more of a polyethylene glycol, a sugar, or a short peptide. In some aspects, the central joint moiety is glutamic acid. In some aspects, the radiopharmaceutical compound is a structure illustrated in Scheme 4.

Furthermore, this disclosure provides a method for in vivo imaging of a disease comprising administering an effective amount of a composition disclosed herein or a compound disclosed herein to a subject. In some aspects the disease is a hepatocarcinoma.

### Discussion

Recent studies have shown that GPC3 plays a critical role on molecular mechanisms by which the proliferation and invasion of HCC are regulated and controlled. The involvement of GPC3 in HCC progression was found through various pathways, including stimulation of Wnt signaling and macrophage recruitment, interaction with growth factors, and promotion of epithelial-mesenchymal transition. It is worthy to note that GPC3 expression levels are significantly different between the tumor tissue in HCCs and the tissue in healthy or nonmalignant livers. For example, in a clinical study, GPC3 expression was detected in 72% of HCC patients, whereas no GPC3 expression was found in patients with a healthy or benign liver. In addition, GPC3 expression was identified in 63-91% of HCC patients in approximate 20 clinical studies. As a result, GPC3 has been considered a valuable biomarker for HCC diagnosis and therapy.

As the development of GPC3-targeted therapies continues to be a very active field of HCC treatment, studying GPC3-targeted PET imaging probes as companion diagnostics has become of great interest. A GPC3-targeted PET probe can be used to noninvasively monitor the GPC3 expression during the tumorigenesis and HCC development, and guide the GPC3-targeted treatment. In addition, due to overexpression of GPC3 in early staged HCCs and minimal GPC3 expression in cirrhotic tissue, a GPC3-targeted PET probe could be useful in distinguishing the early-staged HCC from a benign cirrhotic nodule, which remains a clinical challenge on the HCC diagnosis.

We prepared a peptide-based PET probe for imaging GPC3 expression in HepG2 tumors. Although HepG2 tumors can be visualized by PET, the T/L ratio was low (0.93±0.16) at 1 h pi. Predominant hepatobiliary excretion of the probe causes high radioactivity background in liver, which may hamper the detection of intrahepatic tumor as well as tumor in the abdomen. Low radioactivity background in liver is preferred for a PET probe to be sensitive enough to detect HCCs and/or hepatic metastases. Efforts have been made in the development of PET probes to reduce hepatobiliary excretion and decrease the radioactivity level in liver. One of effective approaches is to increase the hydrophilicity of PET probes by incorporating hydrophilic auxiliaries, such as a carbohydrate moiety, a polyethylene glycol (PEG) unit, and a peptide-based linker. For instance, a linker with six hydrophilic amino acids (GGGRDN) (SEQ ID NO: 3) containing no net charge was introduced to modify F-18 labeled GRPR agonists and antagonists. Incorporating this linker into PET probes takes advantages of 1) an oligo-glycine moiety to facilitate radiolabeling by reducing steric hindrance; 2) an Arg-Asp pair with opposite charges to increase hydrophilicity; and 3) an Asn to serve as a hydrophilic spacer. (See Bioconjugate Techniques, 3^{rd} edition, Greg Hermanson, Academic Press, Aug. 19, 2013).

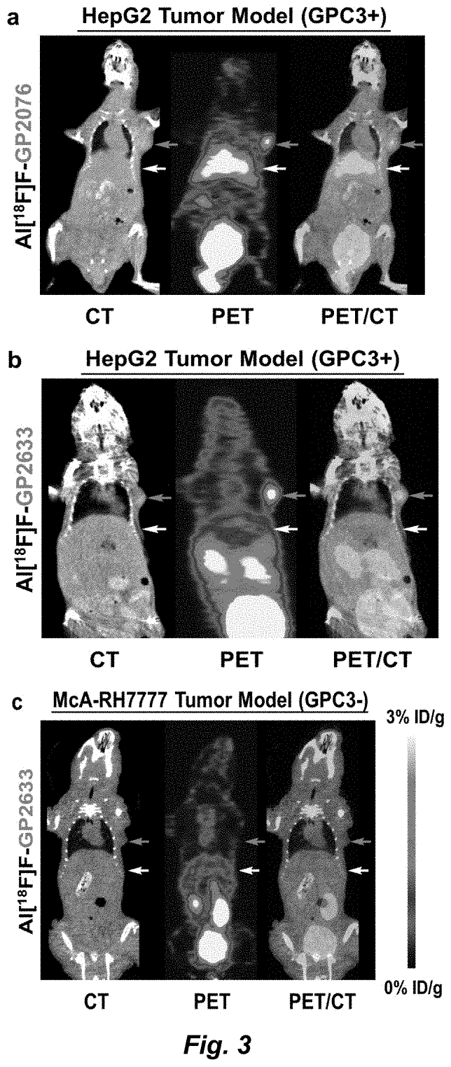

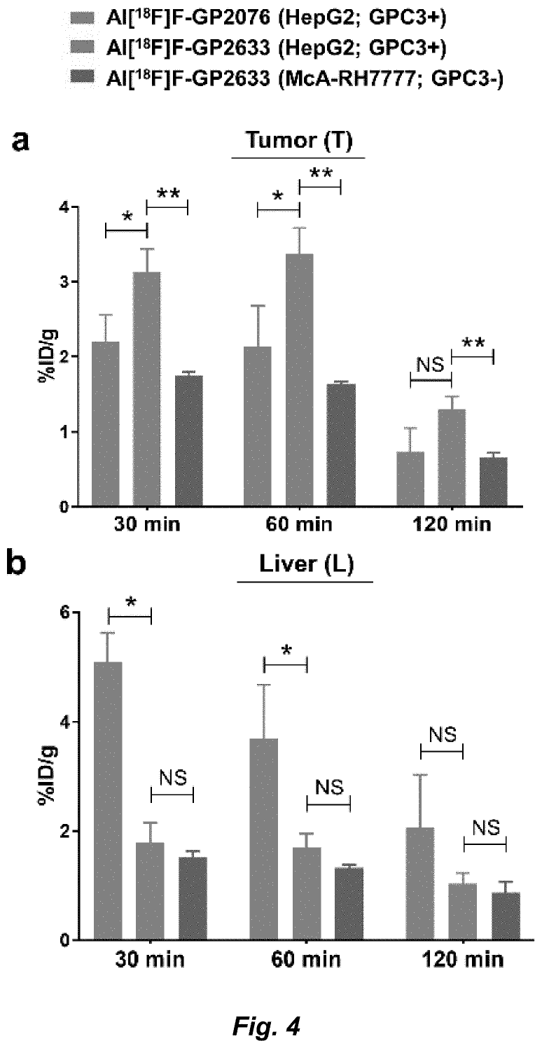

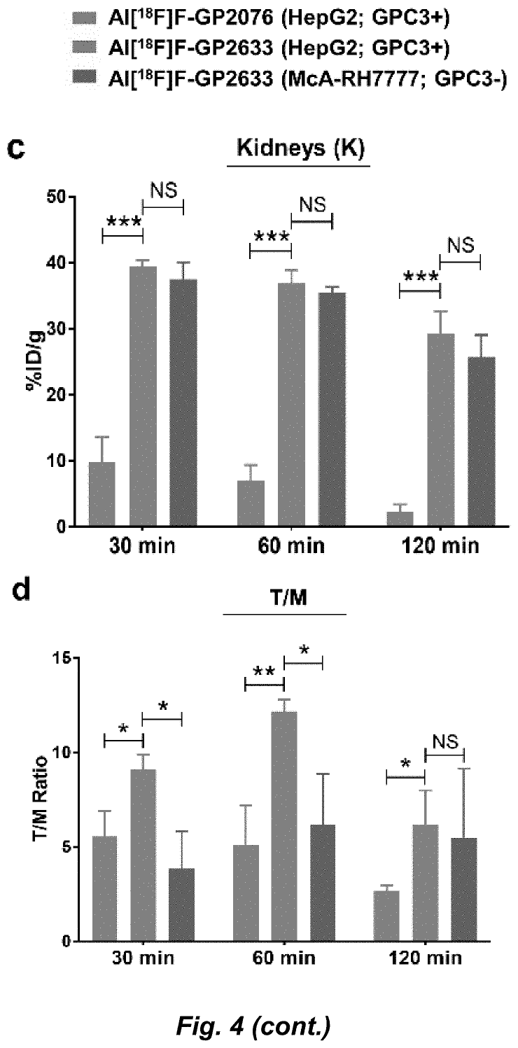

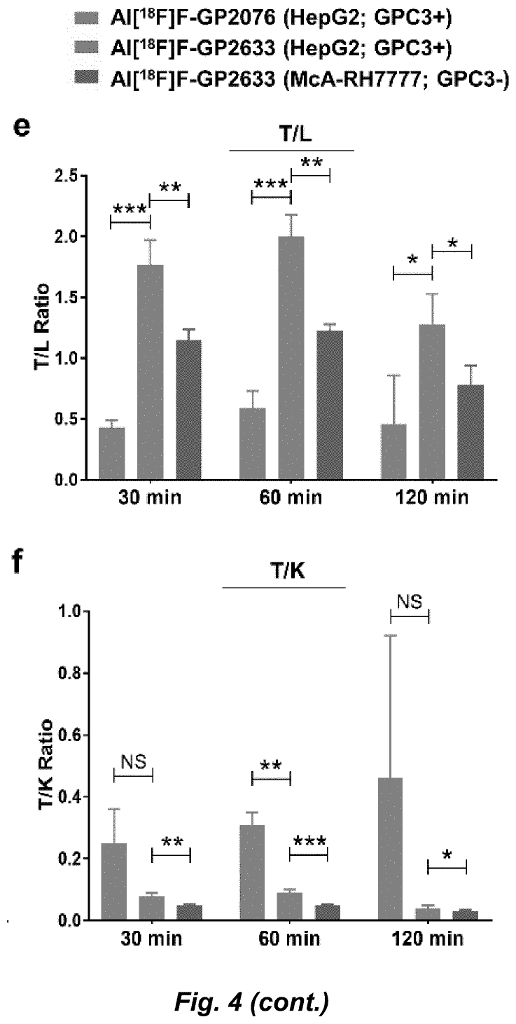

In the present study, a hydrophilic linker of GGGRDN (SEQ ID NO: 3) was conjugated to the GPC3-targeted TP to form a new PET probe (Al[¹⁸F]F-GP2633). The binding assay showed that the addition of the linker slightly enhances the GPC3 binding affinity. The retention time on analytical HPLC and octanol/water partition coefficient confirmed that Al[¹⁸F]F-GP2633 is more hydrophilic than Al[¹⁸F]F-GP2076, a PET probe without the hydrophilic linker. In addition, Al[¹⁸F]F-GP2633 showed excellent specificity of GPC3 binding at a cellular level, and good stability in vitro and in vivo. As compared to Al[¹⁸F]F-GP2076, Al[¹⁸F]F-GP2633 significantly reduced hepatobiliary excretion and achieved a higher T/L ratio for PET imaging at all measured time points (30, 60, and 120 min) (FIGS. 3 and 4). In addition, slightly increased uptake of Al[¹⁸F]F-GP2633 in GPC3-positive HepG2 tumors was also observed as compared to Al[¹⁸F]F-GP2076 (FIG. 4). As expected, Al[¹⁸F]F-GP2633 showed minimal uptake in GPC3-negative McA-RH7777 tumors. The immunohistochemistry analyses confirmed the GPC3 expression levels in HepG2 and McA-RH7777 tumors, which are consistent with the results from PET imaging.

For the radiosynthesis of Al[¹⁸F]F-GP2633, a single-step method was achieved by using the Al[¹⁸F]F chelation approach. The purification of Al[¹⁸F]F-GP2633 without HPLC further simplifies the radiosynthesis procedure. The results demonstrated that the radiosynthesis of Al[¹⁸F]F-GP2633 was simple, fast, and efficient with a good specific activity of the final product.

Overall, this study demonstrated that Al[¹⁸F]F-GP2633 is a GPC3-specific probe with favorable PK for PET imaging in HCC. Convenient preparation, excellent GPC3 specificity in HCC, and promising excretion profile of Al[¹⁸F]F-GP2633 warrant further translational studies.

The new F-18 labeled GPC3-targeted peptides have been successfully developed for PET imaging of GPC3 expression in HCC bearing mice. The PET probe (Al[¹⁸F]F-GP2633) with a hydrophilic linker exhibited better binding affinity to GPC3, enhanced HepG2 tumor uptake, and improved T/L contrast, as compared to the probe (Al[¹⁸F]F-GP2076) without the hydrophilic linker. The preclinical data in this study demonstrated that Al[¹⁸F]F-GP2633 is a promising PET probe for future clinical translation. PET imaging with a GPC3-specific probe would allow clinicians to early detect GPC3-targeted HCC as well as accurately assess tumor response to GPC3-targeted therapy.

The following Examples are intended to illustrate the above invention and should not be construed as to narrow its scope. One skilled in the art will readily recognize that the Examples suggest many other ways in which the invention could be practiced. It should be understood that numerous variations and modifications may be made while remaining within the scope of the invention.

### EXAMPLES

### Example 1

### Radiofluorinated GPC3-Binding Peptides

Hepatocellular carcinoma (HCC) remains one of the most challenging diseases worldwide. Glypican-3 (GPC-3) is a cell surface proteoglycan that is overexpressed on the membrane of HCC cells. The purpose of this study was to develop a target-specific radiofluorinated peptide for positron emission tomography (PET) imaging of GPC3 expression in hepatocellular carcinoma. New GPC3-binding peptides (GP2076 and GP2633) were radiolabeled with F-18 using Al[¹⁸F]F labeling approach, and the resulting PET probes were subsequently subject to biological evaluations. A highly hydrophilic linker was incorporated into GP2633 with an aim of reducing the probe uptake in liver and increasing tumor-to-liver (T/L) contrast. Both GP2076 and GP2633 were radiolabeled using Al[¹⁸F]F chelation approach. The binding affinity, octanol/water partition coefficient, cellular uptake and efflux, and stability of both F-18 labeled peptides were tested. Tumor targeting efficacy and biodistribution of Al[¹⁸F]F-GP2076 and Al[¹⁸F]F-GP2633 were determined by PET imaging in HCC tumor-bearing mice. Immunohistochemistry analyses were performed to compare the findings from PET scans.

Al[¹⁸F]F-GP2076 and Al[¹⁸F]F-GP2633 were rapidly radiosynthesized within 20 min in excellent radiochemical purity (>97%). Al[¹⁸F]F-GP2633 was determined to be more hydrophilic than Al[¹⁸F]F-GP2076 in terms of octanol/water partition coefficient. Both Al[¹⁸F]F-GP2076 and Al[¹⁸F]F-GP2633 demonstrated good in vitro and in vivo stability, and binding specificity to GPC3-positive HepG2 cells. For PET imaging, Al[¹⁸F]F-GP2633 exhibited enhanced uptake in HepG2 tumor (%ID/g: 3.37±0.35 vs. 2.13±0.55, P=0.031) and reduced accumulation in liver (%ID/g: 1.70±0.26 vs. 3.70±0.98, P=0.027) at 60 min post-injection (pi) as compared to Al[¹⁸F]F-GP2076, resulting in significantly improved tumor-to-liver (T/L) contrast (Ratio: 2.00±0.18 vs. 0.59±0.14, P=0.0004). Higher uptake of Al[¹⁸F]F-GP2633 in GPC3-positive HepG2 tumor was observed as compared to GPC3-negative McA-RH7777 tumor (% ID/g: 3.37±0.35 vs. 1.64±0.03, P=0.001) at 60 min pi, confirming GPC3 specific accumulation of Al[¹⁸F]F-GP2633 in HepG2 tumor.

The results demonstrated that Al[¹⁸F]F-GP2633 is a promising probe for PET imaging of GPC3 expression in HCC. Convenient preparation, excellent GPC3 specificity in HCC, and favorable excretion profile of Al[¹⁸F]F-GP2633 warrant further investigation for clinical translation. PET imaging with a GPC3-specific probe would provide clinicians with vital diagnostic information that could have a significant impact on the management of HCC patients.

### Materials and Methods

All chemicals were purchased from commercial suppliers and used without further purification. The peptides RLNVGGTYFLTTRQ (SEQ ID NO: 1) and GGGRDNRLNVGGTYFLTTRQ (SEQ ID NO: 2) were synthesized by the ChinaPeptides Company (Shanghai, China).

### Radiosynthesis and In Vitro Studies

The radiolabeling of the GP2076 or GP2633 peptide was carried out using Al[¹⁸F]F chelation approach (Scheme 1). In brief, to a 5-ml vial containing 2 mM aluminum chloride (6 μl), glacial acetic acid (5 μl), and acetonitrile (334 μl) was added 250 μg of peptide (0.12 μmol of GP2076 or 0.09 μmol of GP2633) in 100 μl deionized (DI) water. After a rapid vibration, 40-50 μl of [¹⁸F]fluoride (555-740 MBq) was added into the mixture. The vial was heated at 100° C. for 10 min. After cooling to room temperature, the mixture was diluted with 15 ml of DI water. The mixture was then passed through a Varian Bond Elut C₁₈ column, and the column was followed by washing with 10 ml of PBS and 20 ml of water. Then 0.4 ml of ethanol containing 10 mM of HCl was used to elute the product. After dilution with saline, the solution passed through a sterile filter, and collected directly into a sterile product vial. The radiochemical purify of Al[¹⁸F]F-GP2076 or Al[¹⁸F]F-GP2633 was determined based on reverse-phase analytical HPLC.

In Vivo Metabolic Stability. HepG2 tumor-bearing mice (n=3/group) were intravenously injected with 5.55 MBq of Al[¹⁸F]F-GP2076 or Al[¹⁸F]F-GP2633. At 1 h after the injection, the mice were euthanized, and the HepG2 tumor, blood, liver, kidneys, and urine samples were collected. Briefly, the blood sample was immediately centrifuged for 5 min at 12,000 rpm. The HepG2 tumor, liver, and kidneys were homogenized and then centrifuged for 5 min at 12,000 rpm. The supernatant from each sample was passed through an ultrafiltration tube (Millipore, USA) and then centrifuged for 10 min at 12,000 rpm. The urine sample was diluted with 100 μl of PBS. The filtrate from each sample was injected into analytic HPLC. The HPLC eluents were collected with a fraction collector (one fraction/30 sec), and the radioactivity of each fraction was measured by gamma counting.

MicroPET/CT Imaging and Biodistribution. MicroPET/CT scans were carried out using a Siemens Inveon PET/CT scanner (Siemens, Germany). Tumor-bearing mice (n=3/group) were intravenously injected with 5.55 MBq of Al[¹⁸F]F-GP2076 or Al[¹⁸F]F-GP2633 under isoflurane anesthesia. A ten-minute static PET scan for each animal was acquired at 30, 60, and 120 min after the injection. 3-Dimensional ordered subset expectation maximization (3D-OSEM) algorithm was used for the PET reconstruction, and CT was applied for attenuation correction. Detailed procedures for the microPET/CT imaging and biodistribution are provided in ESM.

Tumor Histopathology. The tumor (HepG2 or McA-RH7777) tissues were fixed in paraformaldehyde (4%) for 24 h. The specimens were then dehydrated in ethanol, embedded in paraffin, and cut into thick sections (5 μm). The fixed sections were deparaffinized and hydrated according to a standard protocol and stained with hematoxylin and eosin (H&E) for observation. For analysis of GPC3 expression, sections were incubated with an anti-GPC3 antibody at a dilution of 1:150 at 4° C. overnight, and then incubated with a secondary antibody (K5007, polymer-HRP, DAKO, Denmark) at room temperature for 50 min.

### Statistical Analysis

Quantitative data are reported as mean ±standard deviation (SD). Means were compared using one-way ANOVA and student's t-test. All tests were performed using SPSS version 20.0 (IBM Corporation, Armonk, NY, USA). A P value of less than 0.05 was considered statistically significant, and the data were marked with (*) for P<0.05, (**) for P<0.01, and (***) for P<0.001, respectively.

### Results

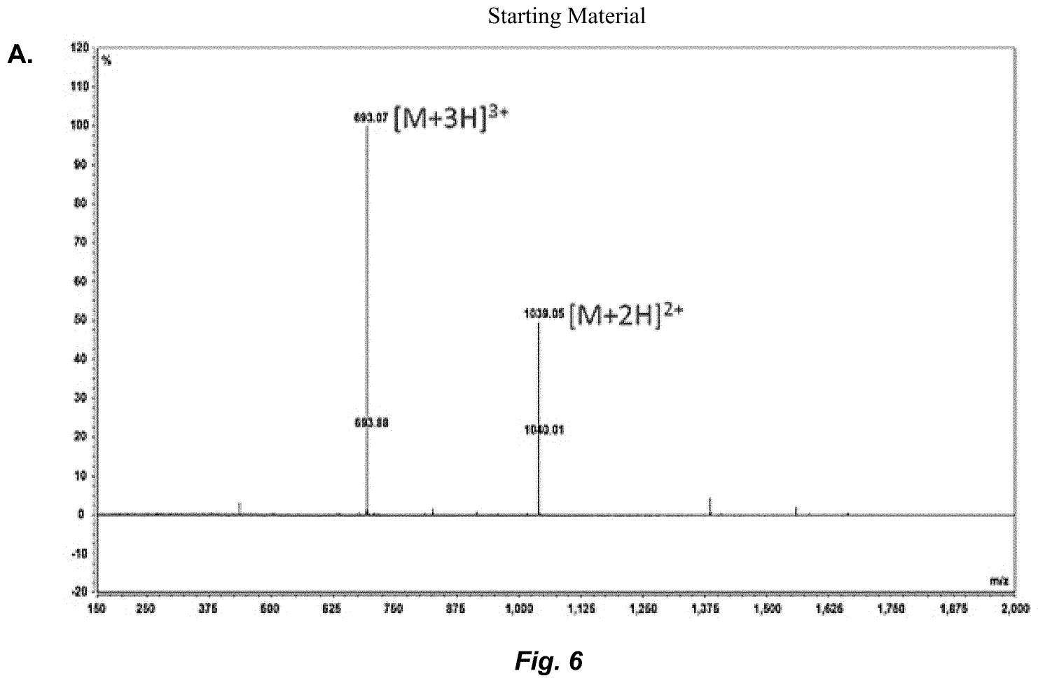

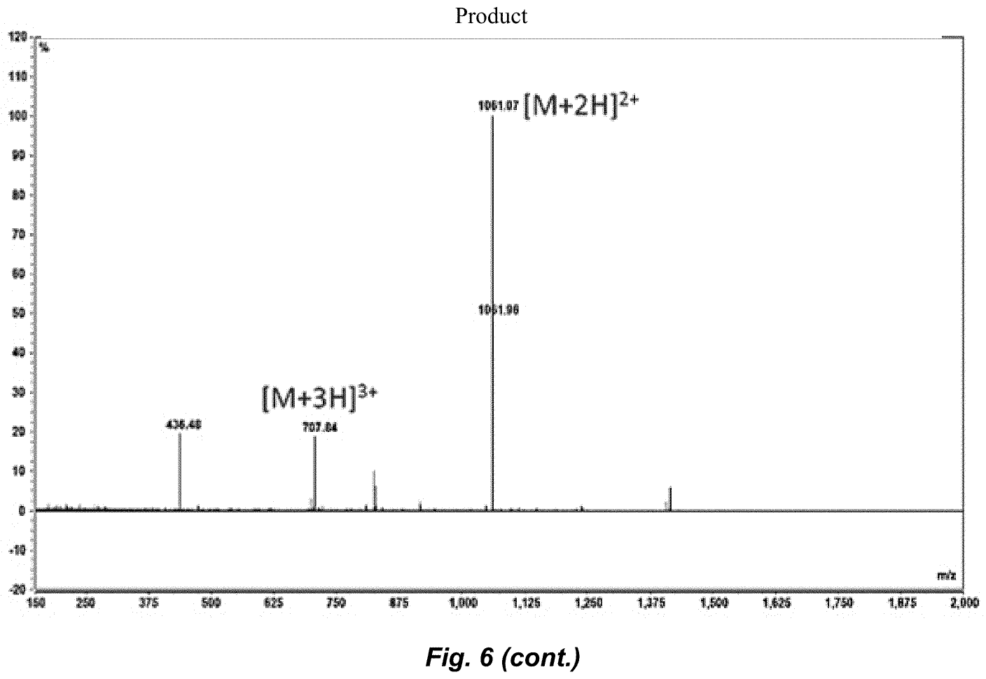

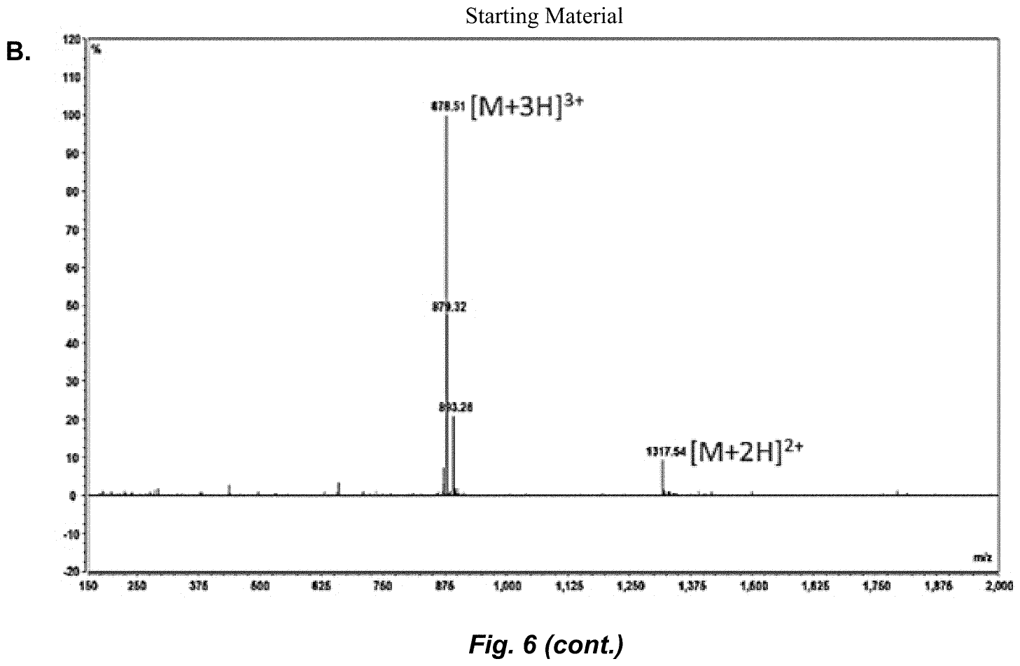

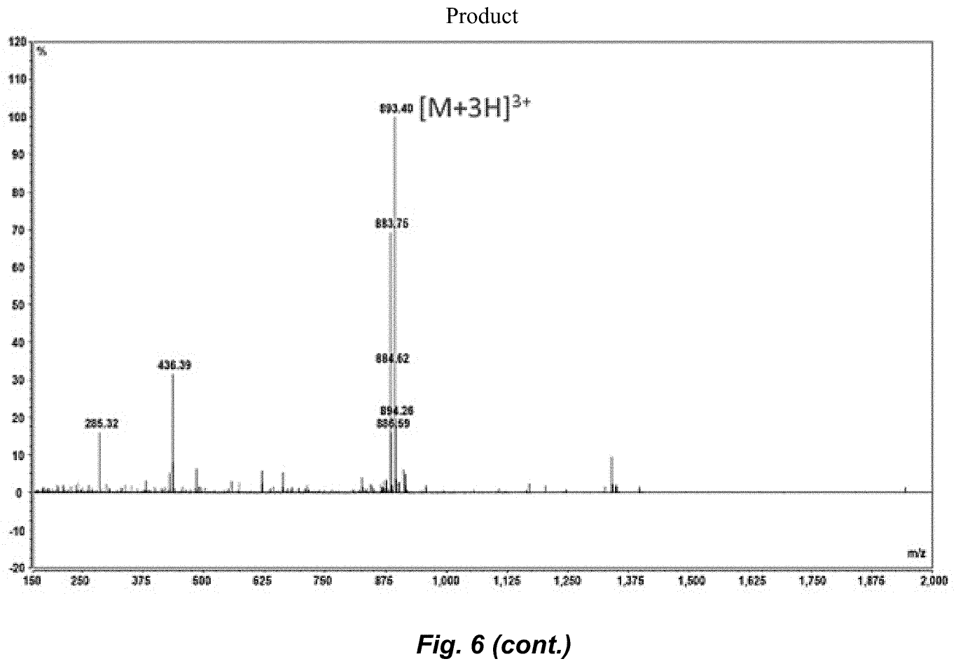

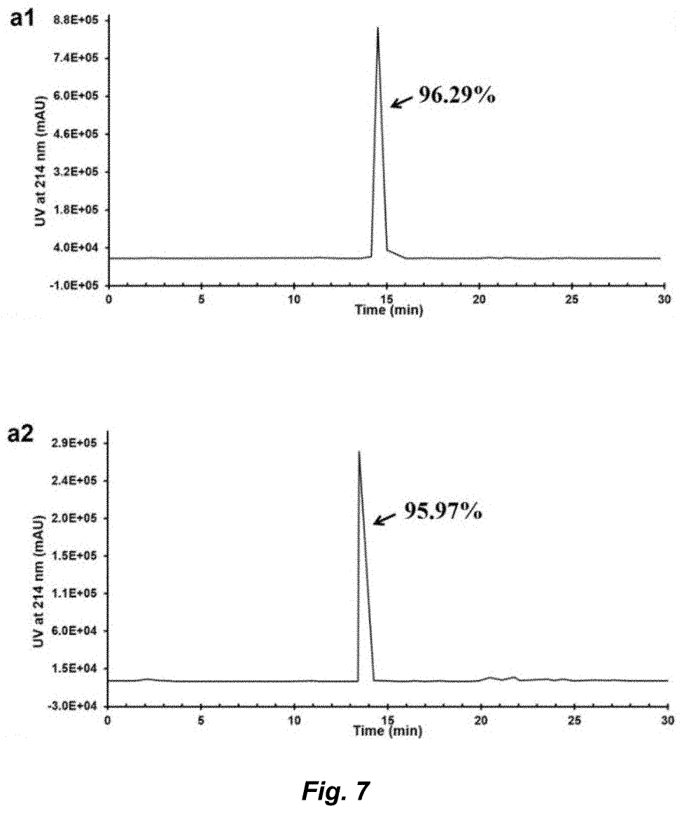

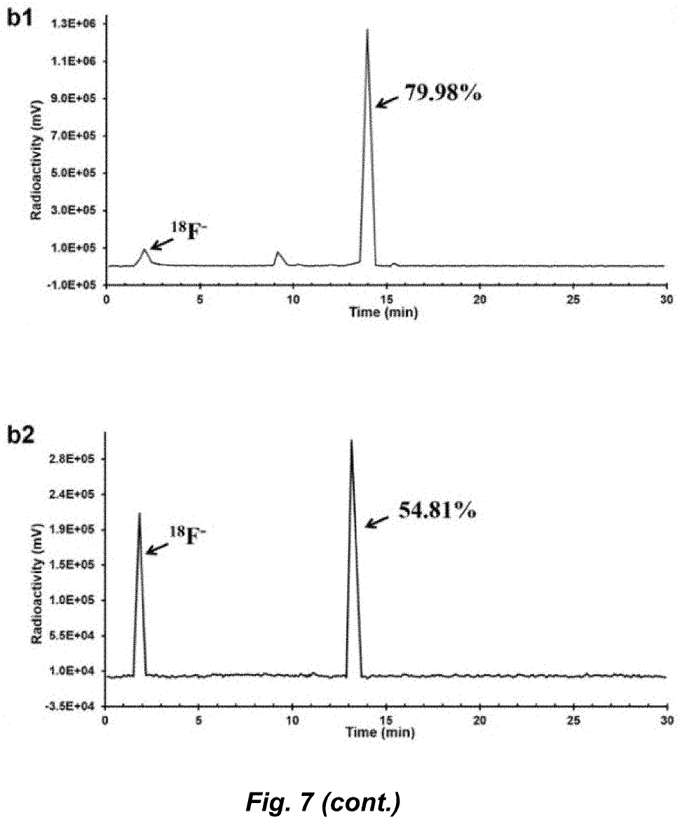

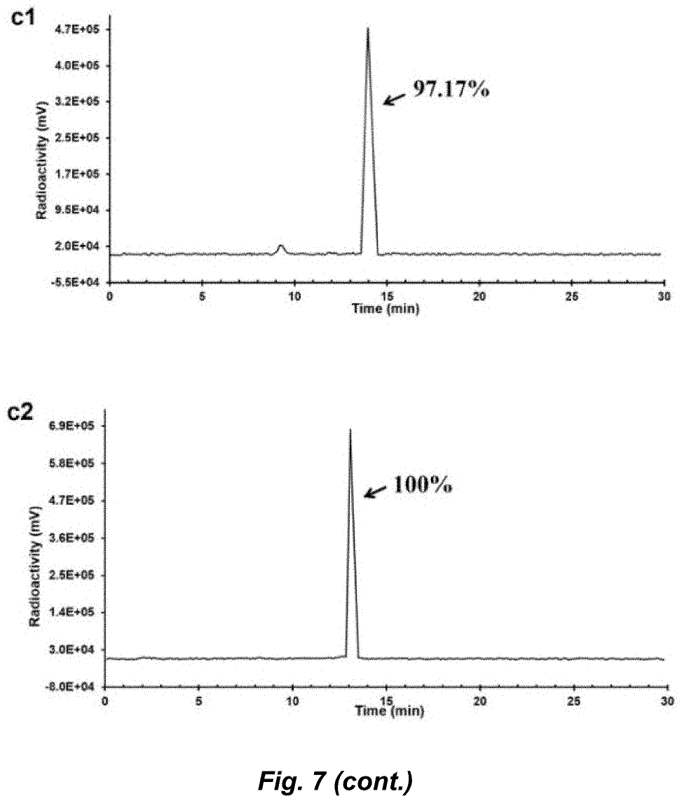

Chemistry and Radiochemistry. Synthetic methods for GP2076, GP2633, and their corresponding Al¹⁹F-labeled peptides are detailed in ESM (Scheme 1). All peptides were obtained in good yield and characterized by mass spectrometry (FIG. 6). Under the identical analytical HPLC condition, the retention time of GP2076 was 14.5 min, while the retention time of GP2633 was 13.5 min (FIGS. 7 a1 and a2).

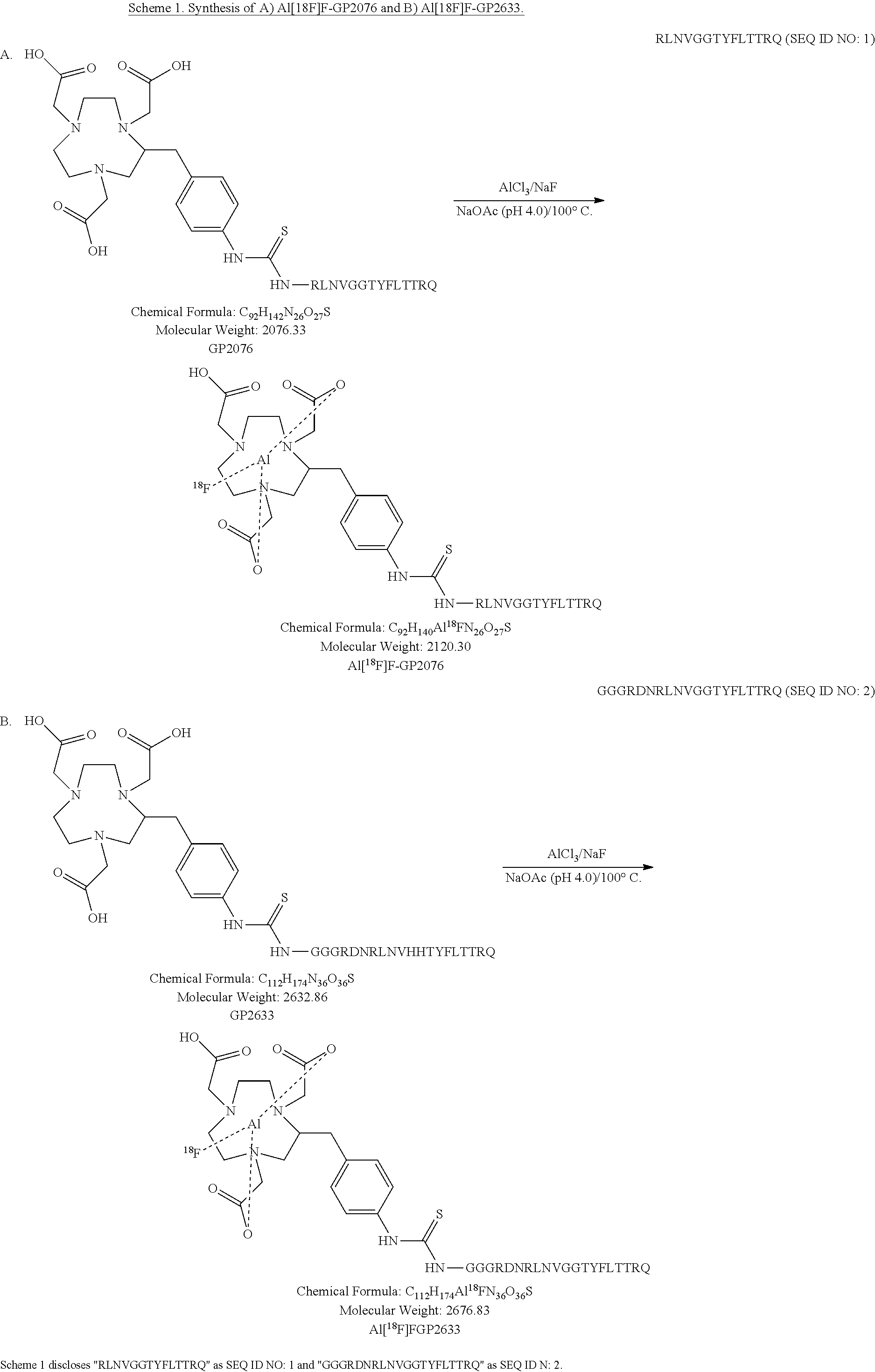

Al[¹⁸F]F-GP2076 and Al[¹⁸F]F-GP2633 were rapidly radiosynthesized using the Al¹⁸F chelation strategy and the solid phase extraction (SPE) purification approach. The total synthesis time for Al[¹⁸F]F-GP2633 and [¹⁸F]Al[¹⁸F]F-GP2076 was approximately 20 min. The radiochemical yields (decay-uncorrected) of Al[¹⁸F]F-GP2076 and Al[¹⁸F]F-GP2633 were 29.25±1.81% (n=3) and 24.86±7.99% (n=3), respectively. After the SPE purification, the radiochemical purities of Al[¹⁸F]F-GP2076 and Al[¹⁸F]F-GP2633 were larger than 97% as determined by analytical HPLC. The retention times of Al[¹⁸F]F-GP2076 and Al[¹⁸F]F-GP2633 were 14.0 min and 13.2 min, respectively (FIG. 7). The specific activities of Al[¹⁸F]F-GP2076 and Al[¹⁸F]F-GP2633 were estimated to be 780.5-1536.5 MBq/μmol and 939.1-1363.8 MBq/μmol, respectively.

The log P values of Al[¹⁸F]F-GP2076 and Al[¹⁸F]F-GP2633 were −2.10±0.07 (n=4) and −2.42±0.09 (n=4), respectively (Table 1), indicating that Al[¹⁸F]F-GP2633 is more hydrophilic than Al[¹⁸F]F-GP2076.

TABLE 1
Lipophilicity (Log P) of Al[¹⁸F]F-GP2076 and Al[¹⁸F]F-GP2633.

| Peptides | Log P* |
|---|---|
| Al[¹⁸F]F-GP2076 | −2.10 ± 0.07 |
| Al[¹⁸F]F-GP2633 | −2.42 ± 0.09 |

*The results are presented as mean ± SD (n = 4/peptide).

Binding Assay. The binding affinity of GP2076 and GP2633 for GPC3 was determined by SPR method. The K_{D} values of GP2076 and GP2633 were calculated to be 101 nM and 63.3 nM, respectively, suggesting that the incorporation of a hydrophilic linker (GGGRDN) (SEQ ID NO: 3) slightly enhances the peptide binding affinity to GPC3.

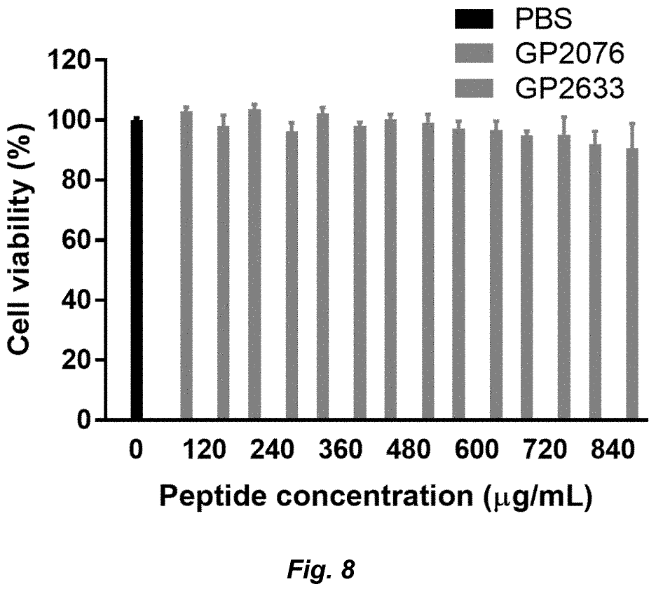

In Vitro Biocompatibility. The cytobiocompatibility of GP2076 and GP2633 was examined prior to in vivo evaluations. As shown in FIG. 8, the cell viabilities of HepG2 cells were larger than 90% at all examined concentrations ranging from 120 to 840 μg/ml, demonstrating the excellent cytocompatibility of GP2076 and GP2633.

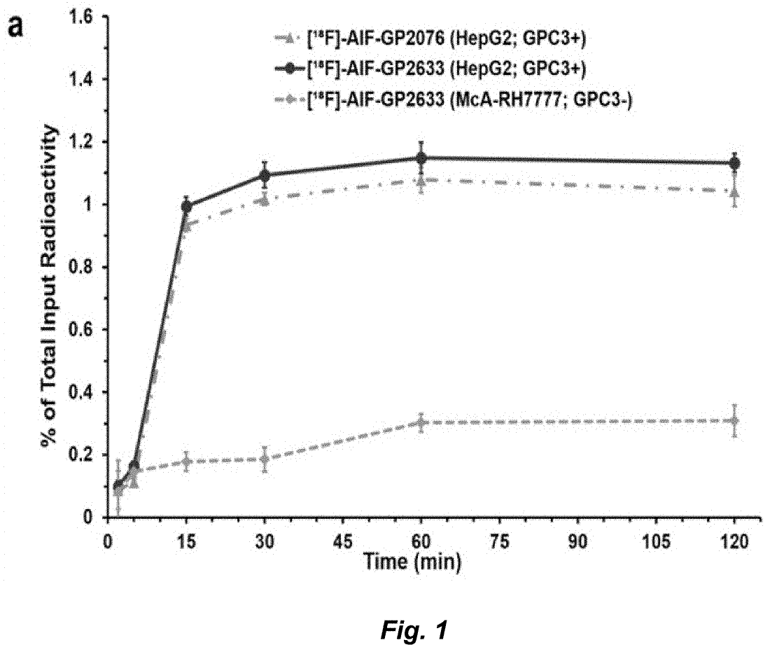

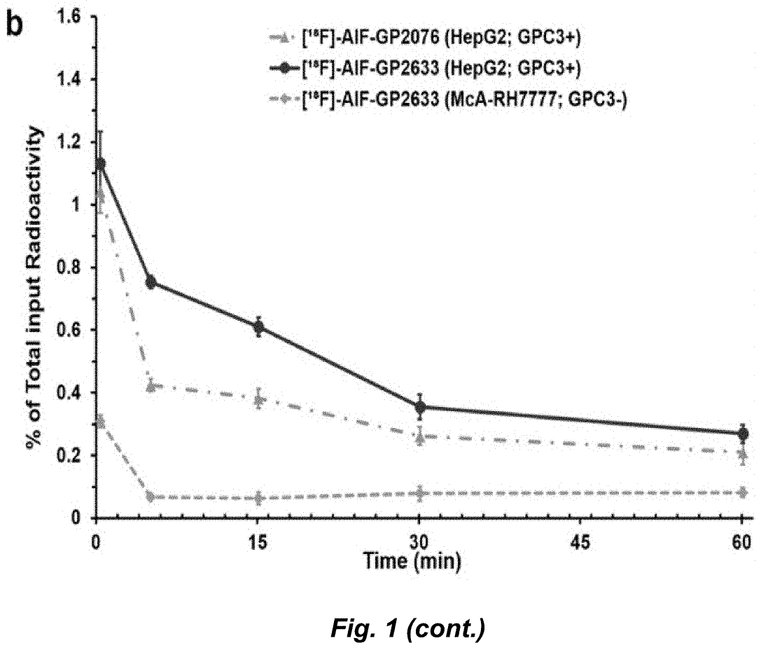

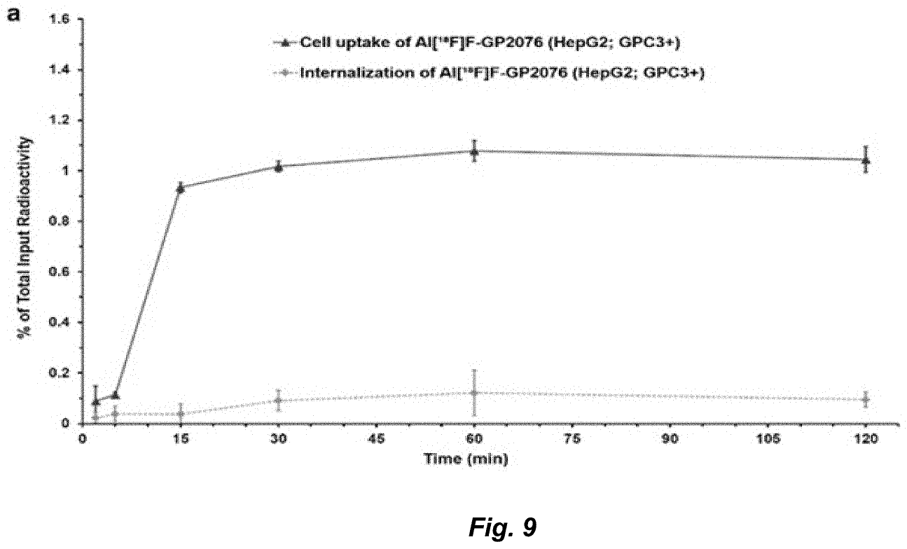

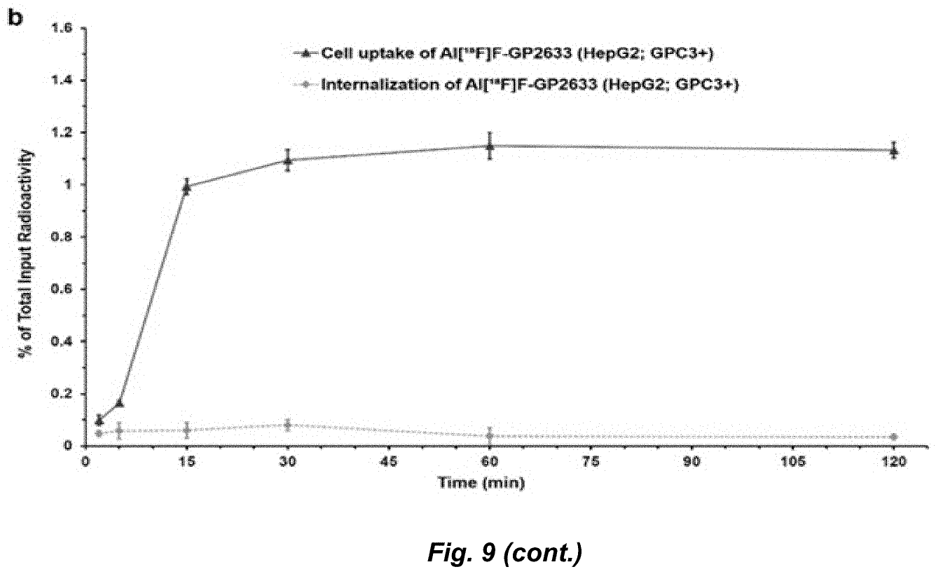

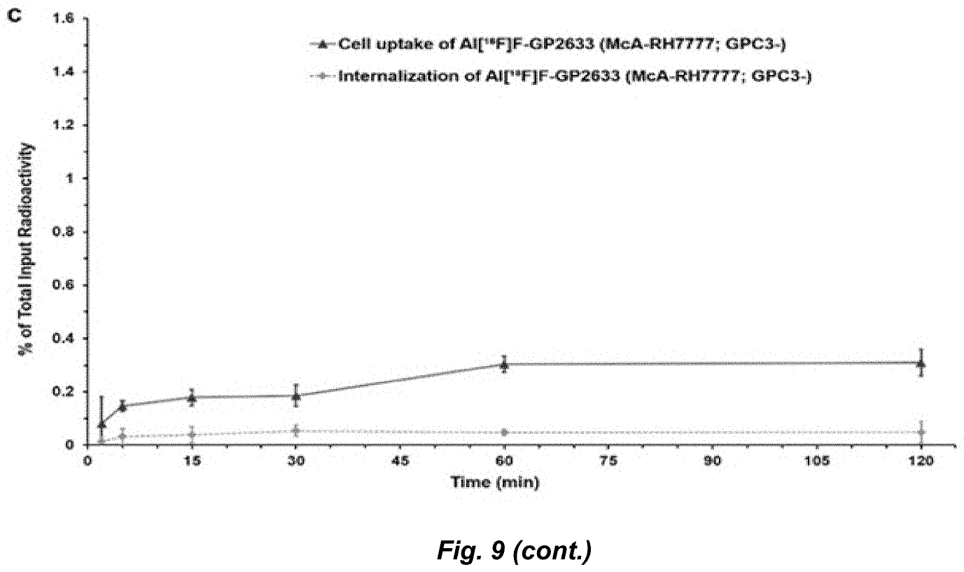

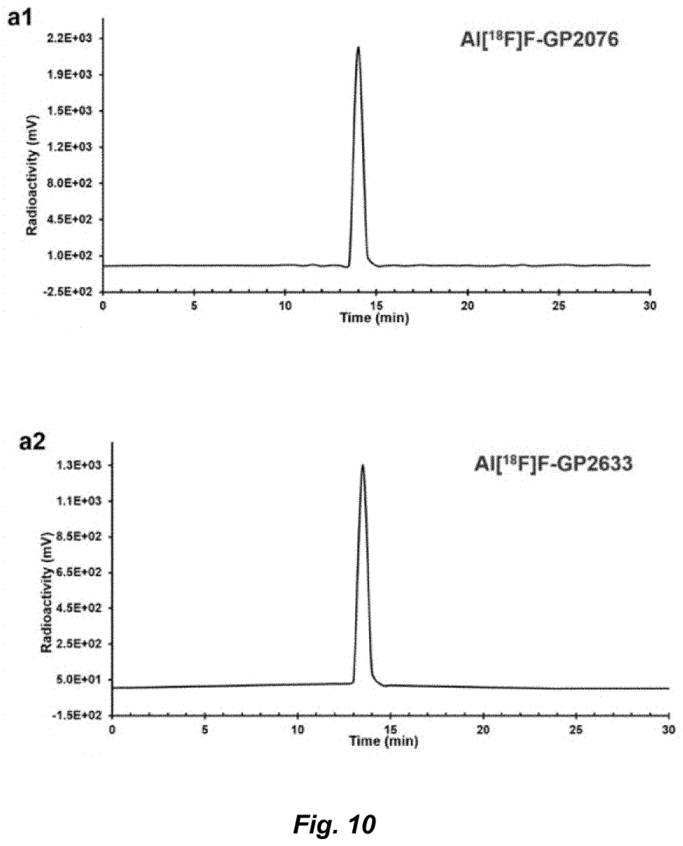

Cellular Uptake, Internalization, and Efflux. The cellular uptake, internalization, and retention of Al[¹⁸F]F-GP2076 and Al[¹⁸F]F-GP2633 were determined in GPC3-positive HepG2 and GPC3-negative McA-RH7777 HCC cells. The cellular uptake results showed that both Al[¹⁸F]F-GP2076 and Al[¹⁸F]F-GP2633 bind to GPC3-positive HepG2 cells very rapidly, and the binding reaches a plateau after 30 min incubation (FIG. 1a). At 60 min, the peak values of cell uptake were 1.08±0.04% for Al[¹⁸F]F-GP2076 and 1.15±0.05% for Al[¹⁸F]F-GP2633, respectively. No significant uptake difference (P=0.721) was observed between Al[¹⁸F]F-GP2076 and Al[¹⁸F]F-GP2633 after 1 h incubation, suggesting that both Al[¹⁸F]F-GP2076 and Al[¹⁸F]F-GP2633 can bind to GPC3-positive HepG2 cells very well. On the other hand, in GPC3-negative McA-RH7777 cells, the cellular uptake values of Al[¹⁸F]F-GP2633 were significantly lower than those in GPC3-positive HepG2 cells after 15 min incubation (FIG. 1a). For instance, at 60 min, the cellular uptake of Al[¹⁸F]F-GP2633 in McA-RH7777 cells was 0.30±0.03%, which is significantly lower than the value (1.15±0.05%, P=0.002) in HepG2 cells. For the cell efflux study, both Al[¹⁸F]F-GP2076 and Al[¹⁸F]F-GP2633 exhibits reasonable cell retention than Al[¹⁸F]F-GP2076 in HepG2 cells (FIG. 1b). During the first 30 min, the efflux (off-target) rate of Al[¹⁸F]F-GP2633 was relatively slower than that of Al[¹⁸F]F-GP2076, suggesting that the binding of Al[¹⁸F]F-GP2633 to GPC3 is slightly stronger than that of Al[¹⁸F]F-GP2076. This result is consistent with the data from the GPC3 binding affinity determination. During 1 h study time, about 0.83% (from 1.04% to 0.21%) and 0.86% (from 1.13% to 0.27%) of radioactivity efflux were observed for Al[¹⁸F]F-GP2076 and Al[¹⁸F]F-GP2633, respectively. In GPC3-negative McA-RH7777 cells, Al[¹⁸F]F-GP2633 showed poor cell retention property, and the radioactivity was rapidly washed out to the baseline within 5 min. Taken together, the cell uptake and efflux data demonstrated that the binding of Al[¹⁸F]F-GP2633 to HepG2 cells is target-specific, which is indeed mediated by GPC3. As shown in FIG. 9, the internalization of both Al[¹⁸F]F-GP2076 and Al[¹⁸F]F-GP2633 in HepG2 cells at 2 h was very low (<10% of cell uptake).

In Vitro and In Vivo Stability. The in vitro stability of [Al[¹⁸F]F-GP2076 and Al[¹⁸F]F-GP2633 was determined in PBS at room temperature and mouse serum at 37° C. after 2 h incubation. The stability was measured as a percentage of intact radiotracer according to the HPLC analysis (FIG. 10). Overall, both Al[¹⁸F]F-GP2076 and Al[¹⁸F]F-GP2633 showed excellent stability in PBS and mouse serum. After 2 h incubation, >95% of Al[¹⁸F]F-GP2076 and >98% of Al[¹⁸F]F-GP2633 remained intact.

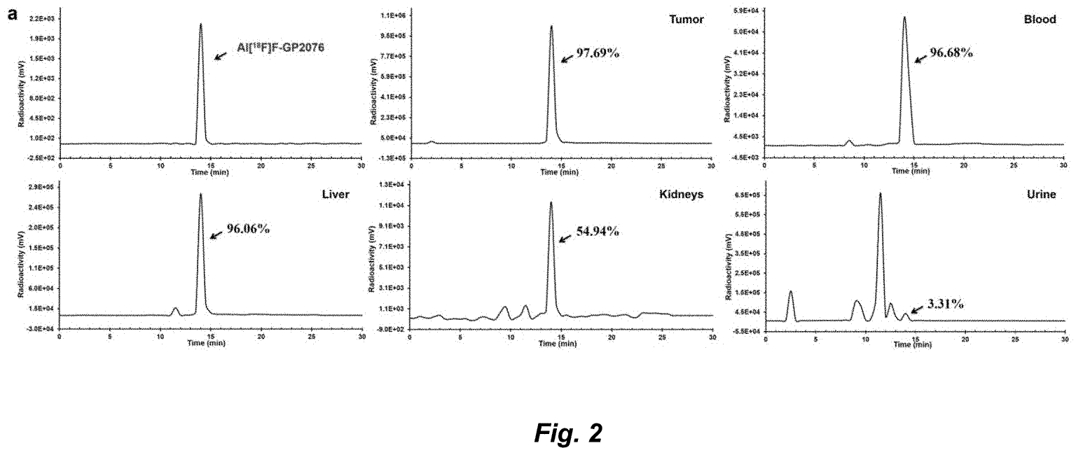

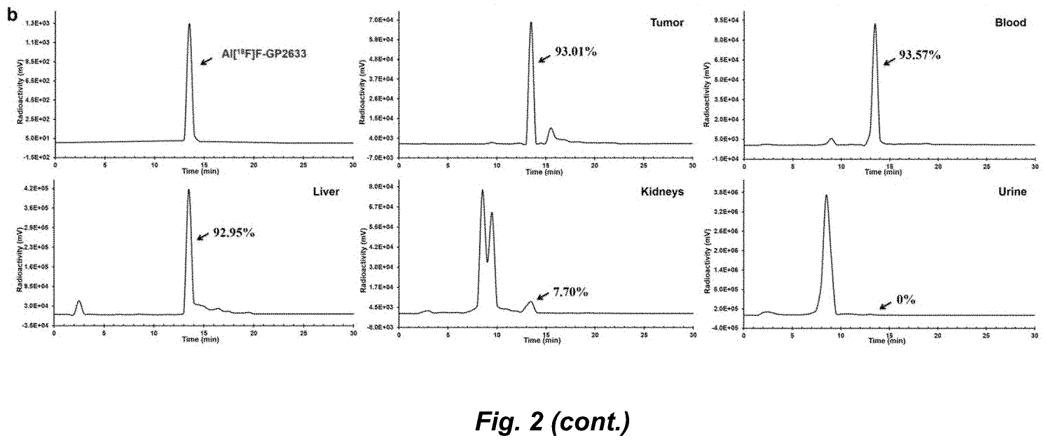

At 1 h after intravenous injection of Al[¹⁸F]F-GP2076 or Al[¹⁸F]F-GP2633 into tumor-bearing mice, the metabolic stability was examined in HepG2 tumor, blood, liver, kidneys, and urine. The samples were analyzed by HPLC, and the representative radioactivity eluent profiles are shown in FIG. 2. For Al[¹⁸F]F-GP2076, the percentage of the parent F-18 labeled peptide was found to 97.69±2.51% in HepG2 tumor, 96.68±1.55% in blood, 96.06±0.54% in liver, 54.94±2.12% in kidneys, and 3.31±0.20% in urine, respectively (FIG. 2a). For Al[¹⁸F]F-GP2633, the percentage of the intact radiotracer was determined to be 93.01±2.98% in HepG2 tumor, 93.57±1.38% in blood, 92.95±2.77% in liver, 7.70±2.56% in kidneys, respectively (FIG. 2b). No parent Al[¹⁸F]F-GP2633 was identified in urine at 1 h post-injection (pi). Overall, both Al[¹⁸F]F-GP2076 and Al[¹⁸F]F-GP2633 displayed similar metabolic stability in HepG2 tumor, blood, and liver. As compared to Al[¹⁸F]F-GP2076, Al[¹⁸F]F-GP2633 was readily catabolized in kidneys, leading to complete metabolite(s) in urine.

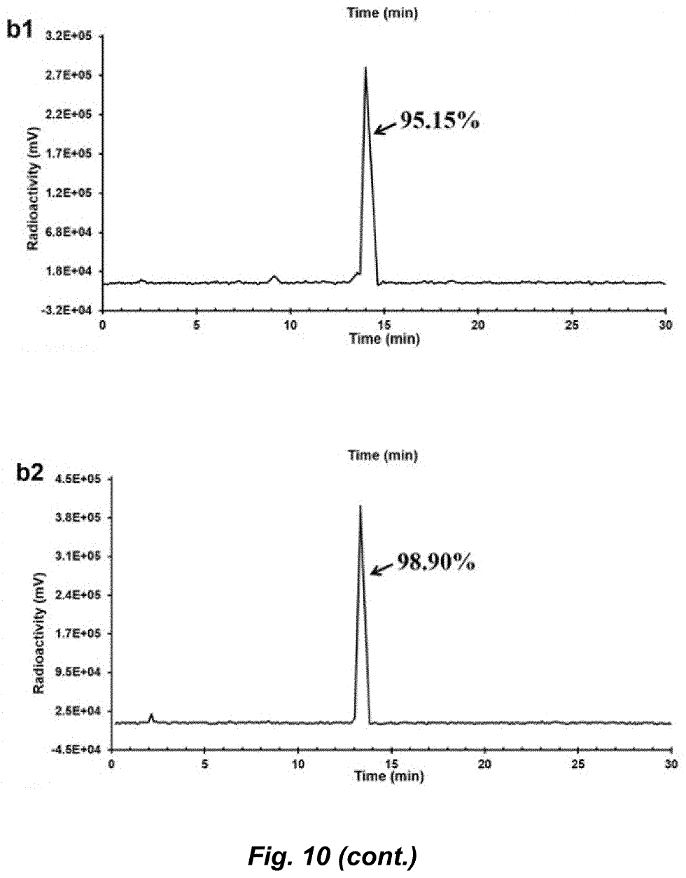

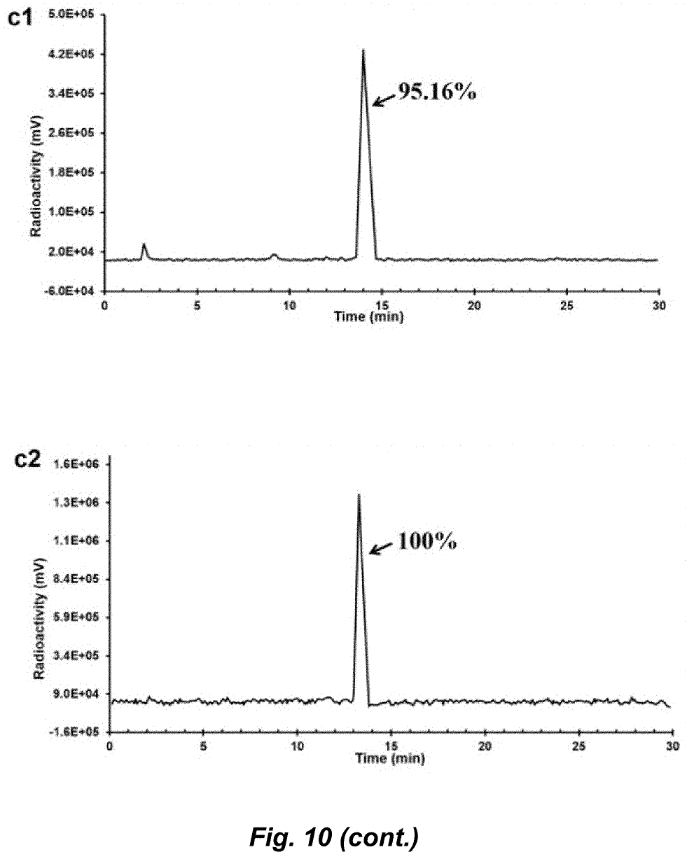

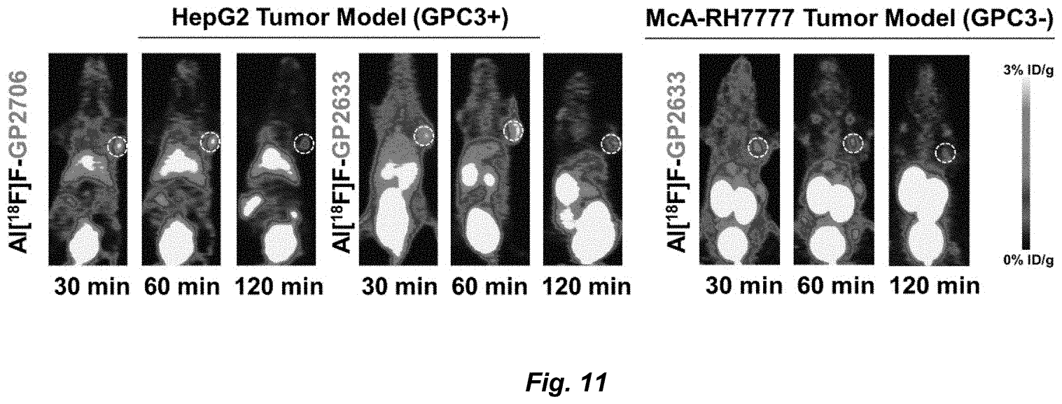

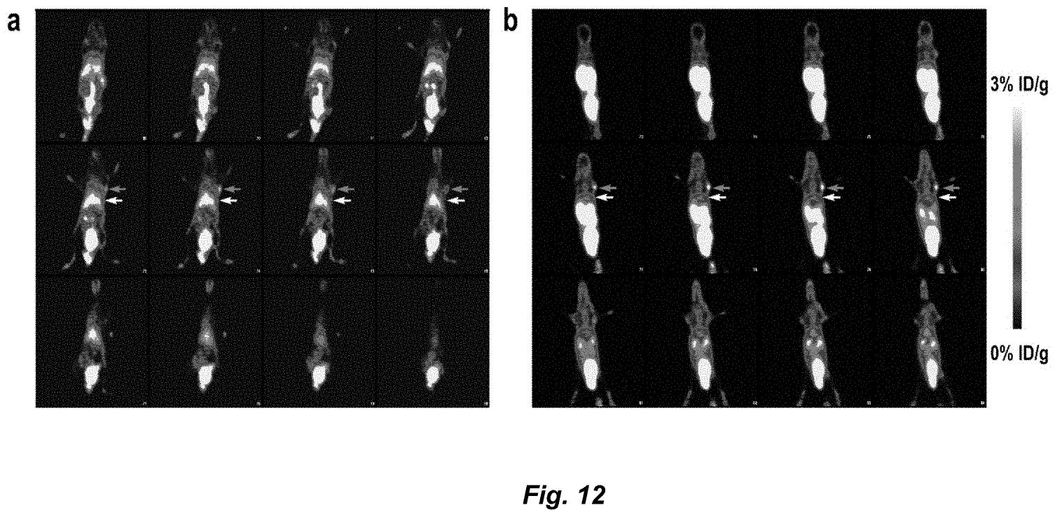

MicroPET/CT Imaging and Biodistribution. The tumor-targeting efficacy and biodistribution of Al[¹⁸F]F-GP2076 and Al[¹⁸F]F-GP2633 were examined in nude mice bearing GPC3-positive HepG2 or GPC3-negative McA-RH7777 tumor xenografts at multiple time points (30, 60, and 120 min) with static PET scans. All GPC3-positive HepG2 tumors were clearly visible at all time points measured after the injection of Al[¹⁸F]F-GP2076 or Al[¹⁸F]F-GP2633; whereas the GPC3-negative McA-RH7777 tumors showed minimal uptake of Al[¹⁸F]F-GP2633. Representative whole-body coronal slices (CT, PET, and PET/CT fusion) containing tumors at 60 min pi are shown in FIG. 3. Representative whole-body coronal PET images of tumor-bearing mice at different time points are presented in FIG. 11. Although both Al[¹⁸F]F-GP2076 and Al[¹⁸F]F-GP2633 exhibited very good uptake in HepG2 tumors, it is worthy to note that the pharmacokinetic (PK) properties of Al[¹⁸F]F-GP2076 and Al[¹⁸F]F-GP2633 are significantly different as visualized by PET images. High liver uptake of Al[¹⁸F]F-GP2076 was observed at all imaging time points, whereas the accumulated radioactivities in liver for Al[¹⁸F]F-GP2633 remained at minimal levels (FIG. 3, FIG. 11, and FIG. 12). Apparently, the clearance of Al[¹⁸F]F-GP2633 from the mouse body is predominantly through the renal system, while the excretion of Al[¹⁸F]F-GP2076 is primarily through the hepatic pathway. The radioactivity accumulated in tumor and major organs was evaluated by measuring the ROIs of the entire organ for each PET scan. The quantitative data of Al[¹⁸F]F-GP2076 and Al[¹⁸F]F-GP2633 at 30, 60, 120 min pi are presented in FIG. 4 and Suppl. Tables 2-14 (see ESM). For the HepG2 tumor uptake, at 30 and 60 min pi, the values of Al[¹⁸F]F-GP2633 were higher than those of Al[¹⁸F]F-GP2076 (% ID/g in HepG2 tumor at 30 min: 3.13±0.31 vs. 2.20±0.36, P=0.027; % ID/g in HepG2 tumor at 60 min: 3.37±0.35 vs. 2.13±0.55, P=0.031). At 120 min pi, the HepG2 tumor uptake values (% ID/g) between Al[¹⁸F]F-GP2076 (0.73±0.32) and Al[¹⁸F]F-GP2633 (1.30±0.17) were not considered statistically significant (P=0.054) (FIG. 4 and Tables 2-3).

For the GPC3-negative McA-RH7777 tumor model, the tumor uptake values of Al[¹⁸F]F-GP2633 (% ID/g: 1.75±0.05 at 30 min, 1.64±0.03 at 60 min, and 0.66±0.06 at 120 min, respectively) were significantly lower than those in the GPC3-positive HepG2 tumor model (% ID/g: 3.13±0.31 at 30 min, 3.37±0.35 at 60 min, and 1.30±0.17 at 120 min, respectively) (FIG. 4 and Tables 2 and 9). For the liver uptake, at 30 and 60 min pi, the values of Al[¹⁸F]F-GP2633 were significantly lower than those of Al[¹⁸F]F-GP2076 (% ID/g in liver at 30 min: 1.80±0.36 vs. 5.10±0.53, P=0.001; % ID/g in liver at 60 min: 1.70±0.26 vs. 3.70±0.98, P=0.027) (FIG. 4 and Tables 2 and 4). For the uptake in kidneys, at 30 and 60 min pi, the values of Al[¹⁸F]F-GP2633 were remarkably greater than those of Al[¹⁸F]F-GP2076 (% ID/g in kidneys at 30 min: 39.40±0.98 vs. 9.83±3.69, P=0.0002; % ID/g in kidneys at 60 min: 36.86±2.05 vs. 7.03±2.32, P=0.0001) (FIG. 4 and Tables 2 and 5).

For the uptake of Al[¹⁸F]F-GP2633 in liver and kidneys, no statistical difference was found between the GPC3-positive HepG2 and GPC3-negative McA-RH7777 tumor model at 30, 60, and 120 min pi (FIG. 4 and Tables 2, 10, and 11). Minimal uptake of Al[¹⁸F]F-GP2076 or Al[¹⁸F]F-GP2633 was found in other major organs, such as brain, heart, and lung. Particularly the low uptake of radioactivity in bone for both Al[¹⁸F]F-GP2076 and Al[¹⁸F]F-GP2633 proved that the Al[¹⁸F]F-NOTA chelation is stable and defluorination from the radiotracers did not occur in vivo.

Based on the quantitative data from the PET scans, the tumor-to-nontarget (T/M, T/L, and T/K) ratios were calculated (FIG. 4 and Tables 6-8 and 12-14). At all measured time points, the values of T/M for Al[¹⁸F]F-GP2633 were significantly greater than those for Al[¹⁸F]F-GP2076 (T/M: 9.11±0.79 vs. 5.56±1.36 (P=0.017) at 30 min, 12.17±0.62 vs. 5.13±2.08 (P=0.005) at 60 min, and 6.20±1.80 vs. 2.68±0.29 (P=0.029) at 120 min, respectively). Significantly higher T/L values were also observed for Al[¹⁸F]F-GP2633 as compared to Al[¹⁸F]F-GP2076 at all measured time points (T/L: 1.77 ±0.20 vs. 0.43±0.06 (P =0.0004) at 30 min, 2.00±0.18 vs. 0.59±0.14 (P=0.0004) at 60 min, and 1.28±0.25 vs. 0.46±0.40 (P=0.039) at 120 min, respectively). At 60 min pi, the T/K value of Al[¹⁸F]F-GP2633 (0.09±0.01) was found to be significantly lower than that of Al[¹⁸F]F-GP2076 (0.31±0.04, P=0.001).

For the GPC3-negative McA-RH7777 tumor model, at 30 and 60 min pi, the T/M ratios of Al[¹⁸F]F-GP2633 (T/M: 3.87±1.97 at 30 min, and 6.20±2.67 at 60 min, respectively) were significantly lower than those in the GPC3-positive HepG2 tumor model (T/M: 9.11±0.79 (P=0.013) at 30 min, and 12.17±0.62 (P=0.019) at 60 min, respectively) (FIG. 4 and Table 12). At 30, 60, and 120 min pi, the T/L and T/K values of Al[¹⁸F]F-GP2633 in the HepG2 tumor model were all significantly higher than those in the McA-RH7777 tumor model (FIG. 4 and Tables 13-14). Overall, at 60 min pi, the best tumor-to-nontarget contrast can be achieved for Al[¹⁸F]F-GP2633 in the GPC3-positive HepG2 tumor model, which can be very well distinguished from two comparing groups: Al[¹⁸F]F-GP2076 in the HepG2 tumor model and Al[¹⁸F]F-GP2633 in the McA-RH7777 tumor model.

The data from the ex vivo biodistribution at 60 min pi are shown in Suppl. Table 15 (see ESM). Overall, the results are consistent with the findings from the PET study. The HepG2 tumor uptake of Al[¹⁸F]F-GP2633 was 1.96±0.29% ID/g which is significantly higher than that of Al[¹⁸F]F-GP2076 (1.13±0.02% ID/g, P=0.007). As compared to Al[¹⁸F]F-GP2076, Al[¹⁸F]F-GP2633 exhibited lower uptake in the liver (0.97±0.07% ID/g vs. 2.30±0.56%ID/g, P=0.015). No statistical difference was observed for the tracer uptake in blood, heart, and bone between the Al[¹⁸F]F-GP2076 and Al[¹⁸F]F-GP2633 groups.

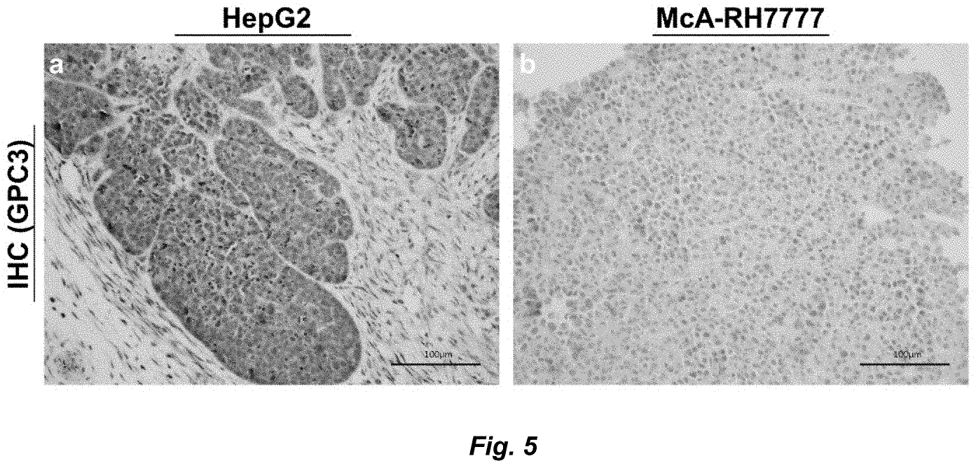

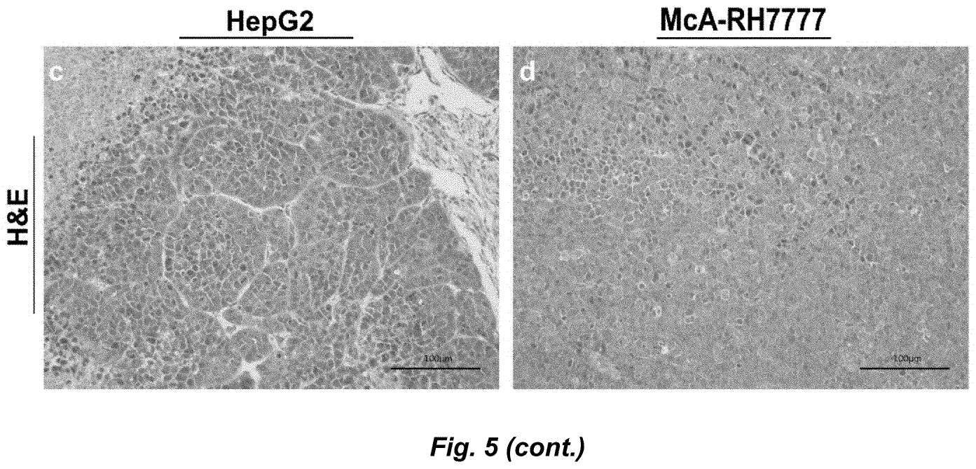

Tumor Histopathology. Qualitative visual assessment of the immunohistochemical assay showed high expression of GPC3 in the HepG2 xenograft (FIG. 5a) whereas the GPC3 expression in McA-RH7777 tumor was minimal (FIG. 5b). Hematoxylin and eosin (H&E) staining demonstrated that no tumor tissue damage was detected in both HepG2 and McA-RH7777 xenograft (FIGS. 5c and 5d).

TABLE 2
Decay-corrected biodistribution of Al[¹⁸F]F-GP2076
or Al[¹⁸F]F-GP2633 in HCC bearing nude mice.*

| | Al[¹⁸F]F-GP2076 | | | Al[¹⁸F]F-GP2633 | | | Al[¹⁸F]F-GP2633 | | |
|---|---|---|---|---|---|---|---|---|---|
| | HepG2 Tumor | | | HepG2 Tumor | | | McA-RH7777 Tumor | | |
| Tissue | Model (GPC3+) | | | Model (GPC3+) | | | Model (GPC3−) | | |
| or Organ | 30 min | 60 min | 120 min | 30 min | 60 min | 120 min | 30 min | 60 min | 120 min |

Percent Injected Dose/gram (% ID/g)

| | | | | | | | | | |
|---|---|---|---|---|---|---|---|---|---|
| Tumor | 2.20 ± | 2.13 ± | 0.73 ± | 3.13 ± | 3.37 ± | 1.30 ± | 1.75 ± | 1.64 ± | 0.66 ± |
| | 0.36 | 0.55 | 0.32 | 0.31 | 0.35 | 0.17 | 0.05 | 0.03 | 0.06 |
| Brain | 0.63 ± | 0.18 ± | 0.13 ± | 0.10 ± | 0.11 ± | 0.22 ± | 0.78 ± | 0.31 ± | 0.22 ± |
| | 0.33 | 0.13 | 0.03 | 0.02 | 0.02 | 0.13 | 0.35 | 0.16 | 0.14 |
| Lung | 1.57 ± | 0.78 ± | 0.43 ± | 0.69 ± | 0.59 ± | 0.34 ± | 0.93 ± | 0.74 ± | 0.49 ± |
| | 0.32 | 0.72 | 0.18 | 0.38 | 0.30 | 0.04 | 0.29 | 0.17 | 0.12 |
| Heart | 1.29 ± | 1.05 ± | 0.36 ± | 1.04 ± | 0.91 ± | 0.37 ± | 1.40 ± | 1.00 ± | 0.47 ± |
| | 0.45 | 0.27 | 0.08 | 0.32 | 0.35 | 0.07 | 0.40 | 0.36 | 0.12 |
| Liver | 5.10 ± | 3.70 ± | 2.07 ± | 1.80 ± | 1.70 ± | 1.04 ± | 1.52 ± | 1.33 ± | 0.88 ± |
| | 0.53 | 0.98 | 0.96 | 0.36 | 0.26 | 0.20 | 0.11 | 0.06 | 0.20 |
| Kidneys | 9.83 ± | 7.03 ± | 2.27 ± | 39.40 ± | 36.86 ± | 29.27 ± | 37.47 ± | 35.40 ± | 25.70 ± |
| | 3.69 | 2.32 | 1.07 | 0.98 | 2.05 | 3.31 | 2.57 | 0.92 | 3.35 |
| Intestinal | 3.03 ± | 2.57 ± | 1.80 ± | 1.09 ± | 1.30 ± | 0.46 ± | 1.43 ± | 1.03 ± | 0.65 ± |
| | 0.91 | 1.16 | 1.07 | 0.31 | 0.10 | 0.38 | 0.49 | 0.25 | 0.23 |
| Muscle | 0.41 ± | 0.44 ± | 0.28 ± | 0.35 ± | 0.28 ± | 0.22 ± | 0.53 ± | 0.31 ± | 0.20 ± |
| | 0.09 | 0.14 | 0.15 | 0.04 | 0.04 | 0.06 | 0.25 | 0.16 | 0.18 |
| Bone | 0.95 ± | 0.35 ± | 0.21 ± | 0.47 ± | 0.43 ± | 0.55 ± | 1.04 ± | 0.80 ± | 0.56 ± |
| | 0.48 | 0.21 | 0.13 | 0.40 | 0.49 | 0.24 | 0.51 | 0.26 | 0.15 |

*The results are presented as mean ± SD (n = 3).

TABLE 3
Decay-corrected HepG2 tumor uptake of Al[¹⁸F]F-GP2076
or Al[¹⁸F]F-GP2633 in HepG2 (GPC3+) tumor
bearing mice (n = 3/group).

| | | Al[¹⁸F]F-GP2076 | Al[¹⁸F]F-GP2633 | | |
|---|---|---|---|---|---|
| Time | | % ID/g (HepG2 (GPC3+) tumor uptake) | | t | P |
| 30 | min | 2.20 ± 0.36 | 3.13 ± 0.31 | 3.421 | 0.027 |
| 60 | min | 2.13 ± 0.55 | 3.37 ± 0.35 | 3.270 | 0.031 |
| 120 | min | 0.73 ± 0.32 | 1.30 ± 0.17 | 2.696 | 0.054 |

TABLE 4
Decay-corrected liver uptake of Al[¹⁸F]F-GP2076 or Al[¹⁸F]F-GP2633
in HepG2 (GPC3+) tumor bearing mice (n = 3/group).

| | | Al[¹⁸F]F-GP2076 | Al[¹⁸F]F-GP2633 | | |
|---|---|---|---|---|---|
| Time | | % ID/g (Liver uptake in HepG2 mice) | | t | P |
| 30 | min | 5.10 ± 0.53 | 1.80 ± 0.36 | 8.927 | 0.001 |
| 60 | min | 3.70 ± 0.98 | 1.70 ± 0.26 | 3.397 | 0.027 |
| 120 | min | 2.07 ± 0.96 | 1.04 ± 0.20 | 1.817 | 0.143 |

TABLE 5
Decay-corrected kidneys uptake of Al[¹⁸F]F-GP2076 or Al[¹⁸F]F-GP2633
in HepG2 (GPC3+) tumor bearing mice (n = 3/group).

| | | Al[¹⁸F]F-GP2076 | Al[¹⁸F]F-GP2633 | | |
|---|---|---|---|---|---|
| Time | | % ID/g (Kidneys uptake in HepG2 mice) | | t | P |
| 30 | min | 9.83 ± 3.69 | 39.40 ± 0.98 | 13.406 | 0.0002 |
| 60 | min | 7.03 ± 2.32 | 36.86 ± 2.05 | 16.663 | 0.0001 |
| 120 | min | 2.27 ± 1.07 | 29.27 ± 3.31 | 13.452 | 0.0002 |

TABLE 6
Tumor-to-muscle (T/M) uptake ratio of Al[¹⁸F]F-GP2076
or Al[¹⁸F]F-GP2633 in HepG2 (GPC3+) tumor
bearing mice (n = 3/group).

| | | Al[¹⁸F]F-GP2076 | Al[¹⁸F]F-GP2633 | | |
|---|---|---|---|---|---|
| Time | | T/M Ratio | | t | P |
| 30 | min | 5.56 ± 1.36 | 9.11 ± 0.79 | 3.920 | 0.017 |
| 60 | min | 5.13 ± 2.08 | 12.17 ± 0.62 | 5.621 | 0.005 |
| 120 | min | 2.68 ± 0.29 | 6.20 ± 1.80 | 3.340 | 0.029 |

TABLE 7
Tumor-to-liver (T/L) uptake ratio of Al[¹⁸F]F-GP2076
or Al[¹⁸F]F-GP2633 in HepG2 (GPC3+) tumor
bearing mice (n = 3/group).

| | | Al[¹⁸F]F-GP2076 | Al[¹⁸F]F-GP2633 | | |
|---|---|---|---|---|---|
| Time | | T/L Ratio | | t | P |
| 30 | min | 0.43 ± 0.06 | 1.77 ± 0.20 | 10.824 | 0.0004 |
| 60 | min | 0.59 ± 0.14 | 2.00 ± 0.18 | 10.679 | 0.0004 |
| 120 | min | 0.46 ± 0.40 | 1.28 ± 0.25 | 3.024 | 0.039 |

TABLE 8
Tumor-to-kidneys (T/K) uptake ratio of Al[¹⁸F]F-GP2076 or
Al[¹⁸F]F-GP2633 in HepG2 (GPC3+) tumor bearing mice (n = 3/group).

| | | Al[¹⁸F]F-GP2076 | Al[¹⁸F]F-GP2633 | | |
|---|---|---|---|---|---|
| Time | | T/K Ratio | | t | P |
| 30 | min | 0.25 ± 0.11 | 0.08 ± 0.01 | 2.686 | 0.055 |
| 60 | min | 0.31 ± 0.04 | 0.09 ± 0.01 | 10.273 | 0.001 |
| 120 | min | 0.46 ± 0.46 | 0.04 ± 0.01 | 1.560 | 0.194 |

TABLE 9
Decay-corrected tumor uptake of Al[¹⁸F]F-GP2633
in HepG2(GPC3+) vs. McA-RH7777 (GPC3−)
tumor bearing mice (n = 3/group).

| | | HepG2 (GPC3+) | McA-RH7777 (GPC3−) | | |
|---|---|---|---|---|---|
| Time | | % ID/g (Tumor uptake) | | t | P |
| 30 | min | 3.13 ± 0.31 | 1.75 ± 0.05 | 7.740 | 0.002 |
| 60 | min | 3.37 ± 0.35 | 1.64 ± 0.03 | 8.497 | 0.001 |
| 120 | min | 1.30 ± 0.17 | 0.66 ± 0.06 | 6.038 | 0.004 |

TABLE 10
Decay-corrected liver uptake of Al[¹⁸F]F-GP2633
in HepG2 (GPC3+) vs. McA-RH7777 (GPC3−) tumor
bearing mice (n = 3/group).

| | | HepG2 (GPC3+) | McA-RH7777 (GPC3−) | | |
|---|---|---|---|---|---|
| Time | | % ID/g (Liver uptake) | | t | P |
| 30 | min | 1.80 ± 0.36 | 1.52 ± 0.11 | 1.287 | 0.268 |
| 60 | min | 1.70 ± 0.26 | 1.33 ± 0.06 | 2.362 | 0.077 |
| 120 | min | 1.04 ± 0.20 | 0.88 ± 0.20 | 0.982 | 0.382 |

TABLE 11
Decay-corrected kidneys uptake of Al[¹⁸F]F-GP2633
in HepG2 (GPC3+) vs. McA -RH7777 (GPC3−) tumor
bearing mice (n = 3/group).

| | | HepG2 (GPP3+) | McA-RH7777 (GPC3−) | | |
|---|---|---|---|---|---|
| Time | | % ID/g (Kidneys uptake) | | t | P |
| 30 | min | 39.40 ± 0.98 | 37.47 ± 2.57 | 1.215 | 0.291 |
| 60 | min | 36.86 ± 2.05 | 35.40 ± 0.92 | 1.130 | 0.322 |
| 120 | min | 29.27 ± 3.31 | 25.70 ± 3.35 | 1.312 | 0.260 |

TABLE 12
Tumor-to-muscle (T/M) uptake ratio of Al[¹⁸F]F-GP2633
in HepG2 (GPC3+) vs. McA-RH7777 (GPC3−) tumor bearing
mice (n = 3/group).

| | | HepG2 (GPP3+) | McA-RH7777 (GPC3−) | | |
|---|---|---|---|---|---|
| Time | | T/M Ratio | | t | P |
| 30 | min | 9.11 ± 0.79 | 3.87 ± 1.97 | 4.291 | 0.013 |
| 60 | min | 12.17 ± 0.62 | 6.20 ± 2.67 | 3.782 | 0.019 |
| 120 | min | 6.20 ± 1.80 | 5.50 ± 3.65 | 0.297 | 0.781 |

TABLE 13
Tumor-to-liver (T/L) uptake ratio of Al[¹⁸F]F-GP2633
in HepG2 (GPC3+) vs. McA -RH7777 (GPC3−) tumor bearing
mice (n = 3/group).

| | | HepG2 (GPP3+) | McA-RH7777 (GPC3−) | | |
|---|---|---|---|---|---|
| Time | | T/L Ratio | | t | P |
| 30 | min | 1.77 ± 0.20 | 1.15 ± 0.09 | 4.733 | 0.009 |
| 60 | min | 2.00 ± 0.18 | 1.23 ± 0.05 | 6.888 | 0.002 |
| 120 | min | 1.28 ± 0.25 | 0.78 ± 0.16 | 2.935 | 0.043 |

TABLE 14
Tumor-to-kidneys (T/K) uptake ratio of Al[¹⁸F]F-GP2633 in HepG2 (GPC3+) vs.
McA-RH7777 (GPC3−) tumor bearing mice (n = 3/group).

| | | HepG2 (GPP3+) | McA-RH7777 (GPC3−) | | |
|---|---|---|---|---|---|
| Time | | T/K Ratio | | t | P |
| 30 | min | 0.08 ± 0.01 | 0.05 ± 0.004 | 6.153 | 0.004 |
| 60 | min | 0.09 ± 0.01 | 0.05 ± 0.002 | 11.040 | 0.0004 |
| 120 | min | 0.04 ± 0.01 | 0.03 ± 0.004 | 3.907 | 0.017 |

TABLE 15
Decay-corrected ex vivo biodistribution of Al[¹⁸F]F-GP2076
or Al[¹⁸F]F-GP2633 in tissues and organs of HepG2
tumor bearing nude mice* at 1 h post-injection.

| | |
|---|---|
| Al[¹⁸F]F-GP2076 | Al[¹⁸F]F-GP2633 |

| | |
|---|---|
| Tissue or Organ | Percent Injected Dose/gram (% ID/g) |

| | | |
|---|---|---|
| HepG2 Tumor | 1.13 ± 0.02 | 1.96 ± 0.29 |
| Brain | 0.10 ± 0.10 | 0.12 ± 0.12 |
| Heart | 0.36 ± 0.15 | 0.39 ± 0.27 |
| Liver | 2.30 ± 0.56 | 0.97 ± 0.07 |
| Kidneys | 6.37 ± 1.45 | 37.05 ± 4.70 |
| Muscle | 0.24 ± 0.07 | 0.31 ± 0.05 |
| Blood | 0.36 ± 0.17 | 0.45 ± 0.12 |
| Bone | 0.29 ± 0.13 | 0.36 ± 0.08 |

*The results are presented as mean ± SD (n = 3).

For further support see Biomaterials (2017) 147:86-98; J Nucl Med (2015) 56:1278-1284; and Eur J Nucl Med Mol Imaging (2007) 34:1823-1831.

### Example 2

### Radiofluorinated GPC3-Binding Peptides for PET Imaging of Hepatocellular Carcinoma

All chemicals were obtained from commercial suppliers and used without further purification. The peptides (sequence: RLNVGGTYFLTTRQ (SEQ ID NO: 1) and GGGRDNRLNVGGTYFLTTRQ (SEQ ID NO: 2)) were synthesized by the ChinaPeptides Company (Shanghai, China). 2, 2′, 2″-(2-(4-isothiocyanatobenzyl)-1,4,7-triazonane-1,4,7-triyl)triacetic acid (p-SCN-Bn-NOTA) was purchased from the AREVA Med Company (Plano, Tex., USA). An anti-GPC3 antibody (Rabbit polyclonal) was obtained from Abcam Company (Shanghai, China). [¹⁸F]Fluoride was produced via the ¹⁸O(p,n)¹⁸F nuclear reaction with a General Electric (GE) PETtrace cyclotron (GE Healthcare, USA). Reverse-phase extraction C18 Sep-Pak cartridges were purchased from Waters (Milford, Mass., USA). The cartridges were pre-conditioned with ethanol and water prior to use. HepG2 hepatocellular carcinoma (HCC) (GPC3-positive) cells and McA-RH7777 HCC (GPC3-negative) cells were obtained from the Institute of Biochemistry and Cell Biology, Shanghai Institutes for Biological Sciences, Chinese Academy of Sciences (Shanghai, China). All animal experiments were conducted in accordance with the guideline of the Nanfang Hospital Animal Ethics Committee at the Southern Medical University. Male or female BALB/c nude mice (about 4-6 weeks old) were obtained from the Laboratory Animal Center at Southern Medical University. Details of other materials and the HPLC methods, and detailed procedures for the measurement of lipophilicity (Log P), cell culture and animal models, binding assay, in vitro biocompatibility, cellular uptake studies, and in vitro stability are provided below.

HPLC Methods. Semi-preparative reversed phase high-performance liquid chromatography (HPLC) for GP2076 or GP2633 was performed on a ThermoFisher UltiMate 3000 HPLC system using a Phenomenex Luna C18 reversed phase column (5 μm, 250×10 mm). The flow rate was 4 mL/min, with the mobile phase starting from 95% solvent A (0.1% trifluoroacetic acid (TFA) in water) and 5% solvent B (0.1% TFA in acetonitrile) to 40% solvent A and 60% solvent B during 25.5 min. The UV absorbance was monitored at 214 nm and 254 nm. The radiolabeled peptides were identified using an analytic HPLC system (Shimadzu, Japan) consisting of a Shimadzu LC-LOAD pump, a variable wavelength SPD-M20A UV detector, and a Flow Count radio-HPLC Detector (Bioscan). The UV absorbance was monitored at 214 and 254 nm. The analytic HPLC was performed on a ZORBAX Eclipse XDB-C18 column (5 μm, 150×4.6 mm). The flow rate was 1 mL/min with the mobile phase starting from 95% solvent A (0.1% TFA in water) to 20% solvent A and 80% solvent B (0.1% TFA in acetonitrile) during 25 min.

Synthesis of GP2076. The peptide RLNVGGTYFLTTRQ (SEQ ID NO: 1) (2.0 mg, 1.23 μmol) dissolved in 0.5 mL of sodium borate buffer (pH 8.5) was mixed with p-SCN-Bn-NOTA (0.7 mg, 1.25 μmol) in 20 μL of DMSO. The pH of mixture was adjusted to 8.5 using 0.1 M NaOH. After sonication at 40° C. for 2 h, the mixture was dissolved in water and purified by semi-preparative HPLC. The peak containing the GP2076 peptide was collected and lyophilized to afford fluffy white powder (1.9 mg, yield: 74%). ESI-MS m/z C₉₂H₁₄₂N₂₆O₂₇S: [M+2H]²⁺ calcd, 1039.17, found, 1039.05; [M+3H]³⁺ calcd, 693.11, found, 693.07.

Synthesis of [¹⁹F]-ALF-GP2076. To a 1 mL V-vial containing 0.2 mL of deionized water were added 10 μL of 2 mM aluminum chloride in 0.1 M sodium acetate buffer (pH 4.0) and 7 μL of 3 mM sodium fluoride in 0.1 M sodium acetate buffer (pH 4.0). The mixture was heated at 100° C. for 10 min. To the reaction mixture, 5 μL of 2.5 mM GP2076 in 0.1 M sodium acetate buffer (pH 4.0) was added, and the mixture was heated at 100° C. for additional 10 min. The mixture was cooled and then purified by semi-preparative HPLC. The peak containing the [¹⁹F]-AlF-GP2076 peptide was collected. ESI-MS m/z C₉₂H₁₄₀AlFN₂₆O₂₇S: [M+2H]²⁺ calcd, 1061.15, found, 1061.07; [M+3H]³⁺ calcd, 707.77, found, 707.84.

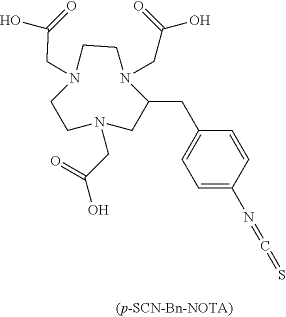 [SMILES: O=C(O)CN1CCN(CC(=O)O)CC(Cc2ccc(N=C=S)cc2)N(CC(=O)O)CC1]

Synthesis of GP2633. The peptide GGGRDNRLNVGGTYFLTTRQ (SEQ ID NO: 2) (2.6 mg, 1.0 μmol) dissolved in 0.5 mL of sodium borate buffer (pH 8.5) was mixed with p-SCN-Bn-NOTA (0.6 mg, 1.07 μmol) in 20 μL of DMSO. The pH of mixture was adjusted to 8.5 using 0.1 M NaOH. After sonication at 40° C. for 2 h, the mixture was dissolved in water and purified by semi-preparative HPLC. The peak containing the GP2633 peptide was collected and lyophilized to afford a fluffy white powder (1.8 mg, yield: 68%). ESI-MS m/z C₁₁₂H₁₇₄N₃₆O₃₆S: [M+2H]²⁺ calcd, 1317.43, found, 1317.54; [M+3H]³⁺ calcd, 878.62, found, 878.51.

Synthesis of [¹⁹1]-AlF-GP2633. To a 1 mL V-vial containing 0.2 mL of deionized water were added 10 μL of 2 mM aluminum chloride in 0.1 M sodium acetate buffer (pH 4.0) and 7 μL of 3 mM sodium fluoride in 0.1 M sodium acetate buffer (pH 4.0). The mixture was heated at 100° C. for 10 min. To the reaction mixture, 5 μL of 2.5 mM GP2633 in 0.1 M sodium acetate buffer (pH 4.0) was added, and the mixture was heated at 100° C. for additional 10 min. The mixture was cooled and then purified by semi-preparative HPLC. The peak containing the [¹⁹F]-AlF-GP2633 peptide was collected. ESI-MS m/z C₁₁₂H₁₇₂AlFN₃₆O₃₆S: [M+3H]³⁺ calcd, 893.28, found, 893.40.

Measurement of Lipophilicity (Log P). Approximately 185 kBq of Al[¹⁸F]F-GP2076 or Al[¹⁸F]F-GP2633 in 0.5 ml of phosphate-buffered saline (PBS) (pH 7.4) was mixed with 0.5 ml of 1-octanol. The mixture was vigorously shaken for 1 min, and then centrifuged at 12,000 rpm for 5 min to partition the organic and aqueous layers. Aliquots of 0.2 ml each layer were taken and the radioactivity was determined by gamma counting (GC-1200, USTC Chuangxin Co. Ltd. Zonkia Branch, China). The distribution coefficient P was calculated as the ratio of radioactivity in the organic phase to that in the aqueous phase. The experiment was carried out in quadruplicate.

Cell Culture and Animal Models. HepG2 HCC (GPC3-positive) cells and McA-RH7777 HCC (GPC3-negative) cells were obtained from the Shanghai Institute of Biochemistry and Cell Biology, Chinese Academy of Sciences (Shanghai, China). The cells were cultured in Dulbecco's Modified Eagle's Medium (DMEM) (ThermoFisher Scientific, USA) supplemented with 10% fetal bovine serum (ThermoFisher Scientific, USA). Cells were grown at 37° C. in a humidified atmosphere containing 5% CO₂.

All animal experiments were conducted according to the guideline of the Nanfang Hospital Animal Ethics Committee at the Southern Medical University. Male or female BALB/c nude mice (about 4-6 weeks old) were obtained from the Laboratory Animal Center at Southern Medical University. The HepG2 and McA-RH7777 HCC xenografts were established by subcutaneous injection of 1×10⁶ tumor cells into the left shoulder of nude mice. The animals were used for in vivo studies when the tumors reached a size of 0.5-1 cm in diameter (4-6 weeks after inoculation).

Binding Assay. The binding affinity of the GP2076 or GP2633 peptide for GPC3 was determined using surface plasmon resonance (SPR) measurements (PlexArray HT A100, Plexera, USA). In brief, after the GPC3 protein (Novoprotein, China) was immobilized on a 3D Dextran chip, the GP2076 or GP2633 peptide flowed at increasing concentrations (400 nM and 800 nM). The results were analyzed by PlexeraDE software.

In Vitro Biocompatibility. A colorimetric assay was utilized to determine cell viability after treating GPC3 positive HepG2 cells with the GP2076 or GP2633 peptide. The assay was carried out according to the instruction of the manufacturer. In brief, HepG2 cells were seeded at a density of 1×10⁴ cells/well in a 96-well plate. After the incubation of the GP2076 or GP2633 peptide at various concentrations (0, 120, 240, 360, 480, 600, 720, and 840 μg/ml) with HepG2 cells for 24 h, the HepG2 cells were examined using the cell counting Kit-8 (KeyGen Biotech, Nanjing, China). The absorbance at 450 nm of all the wells in the 96-well microplate was recorded on a microplate reader (BIOTEK ELX800, USA). The experiment was performed in quadruplicate.

Cellular Uptake, Internalization, and Efflux Studies. Cellular uptake and efflux of Al[¹⁸F]F-GP2076 or Al[¹⁸F]F-GP2633 in HepG2 or McA-RH7777 cells were performed according to a previously reported protocol. In the cellular uptake study, HepG2 or McA-RH7777 cells were seeded into 12-well plates at a density of 5×10⁵ cells per well. After overnight incubation, cells were rinsed 3 times with PBS, followed by the addition of Al[¹⁸F]F-GP2076 or Al[¹⁸F]F-GP2633 (0.74-1.11 MBq/well) to the wells in quadruplicate. After incubation at 37° C. for 2, 5, 15, 30, 60, and 120 min, cells were rinsed 3 times with PBS and lysed with 0.2 M NaOH containing 1% sodium dodecyl sulfate (SDS). The radioactivity associated with cell lysate was measured by gamma counting. The cell uptake was normalized by the added radioactivity after decay correction.

The internalization assay was carried out similarly to the procedure of cell uptake study except for an additional wash with acid buffer (50 mM glycine, 0.1 M NaCl, pH 2.8) for 1 min, which was conducted after the two PBS washes in order to remove the membrane-bound Al[¹⁸F]F-GP2076 or Al[¹⁸F]F-GP2633. After the 1 min incubation with the acid buffer, the cells were washed again with cold PBS and removed from the plate. The radioactivity associated with the internalized fraction was measured by gamma counting.

In the cellular efflux study, HepG2 or McA-RH7777 cells were seeded into 12-well plates at a density of 5×10⁵ cells per well. After overnight incubation, cells were rinsed 3 times with PBS, and then incubated with Al[¹⁸F]F-GP2076 or Al[¹⁸F]F-GP2633 (0.74-1.11 MB q/well) at 37° C. for 2 h. After the PBS washing and re-incubation with serum-free medium, cells were then washed at different time points (0, 5, 15, 30, and 60 min) with PBS and lysed with 0.2 M NaOH containing 1% SDS. The radioactivity associated with cell lysate was measured by gamma counting. Cell efflux results are presented as a percentage of the added dose after decay correction.

In Vitro Stability. The stability of Al[¹⁸F]F-GP2076 or Al[¹⁸F]F-GP2633 was evaluated in PBS and mouse serum. Briefly, 5.55 MBq of Al[¹⁸F]F-GP2076 or Al[¹⁸F]F-GP2633 was incubated with 0.5 ml of PBS at room temperature or mouse serum at 37° C. with gentle shaking. The stability test was carried out at 2 h after the incubation. For the PBS study, an aliquot of the solution was taken, and the radiochemical purity was determined by analytical HPLC. For the mouse serum study, after the addition of TFA, the mixture was passed through a 0.2-μm filter. An aliquot of the soluble fraction was taken, and the radiochemical purity was examined by analytical HPLC.

MicroPET/CT Imaging and Biodistribution. MicroPET/CT scans were carried out using a Siemens Inveon PET/CT scanner (Siemens, Germany). Tumor-bearing mice (n=3/group) were intravenously injected with 5.55 MBq of Al[¹⁸F]F-GP2076 or Al[¹⁸F]F-GP2633 under isoflurane anesthesia. A ten-minute static PET scan for each animal was acquired at 30, 60, and 120 min after the injection. 3-Dimensional ordered subset expectation maximization (3D-OSEM) algorithm was used for the PET reconstruction, and CT was applied for attenuation correction. Detailed procedures for the microPET/CT imaging and biodistribution are provided in ESM.

Inveon Research Workplace (IRW) 3.0 software (Siemens, Germany) was used to measure the regions of interest (ROIs) determined by superimposing the ellipsoid volume of interest (VOI) on the target tissues. The radioactivity concentrations were measured by the mean pixel intensity within each VOI and converted to dose/ml using a calibration constant. Assuming the tissue density of 1 g/ml, the ROI was then converted to dose per gram and normalized as the percent injected dose per gram (% ID/g). Tumor-to-nontarget uptake ratios, including tumor-to-muscle (T/M), tumor-to-liver (T/L), and tumor-to-kidneys (T/K) ratios, were calculated by dividing the radioactivity uptake in tumor by that in the corresponding normal tissue or organ.

Ex vivo biodistribution was evaluated at 60 min after the tail vein injection of 1.85 MBq of Al[¹⁸F]F-GP2076 or Al[¹⁸F]F-GP2633. Mice were euthanized and dissected. Major tissues and organs were collected and weighed wet. The radioactivity in the tissues and organs was measured using a gamma counter. The results were presented as % ID/g. For each animal, the radioactivity of the tissue and organ samples was calibrated with a known aliquot of the injected activity. Mean uptake (% ID/g) for a group of animals was calculated with standard deviations.

### Example 3

### Radiofluorinated GPC3-Binding Dimer Peptides

Scheme 2 illustrates the formation of certain radiopharmaceutical compound comprising two or more linear peptides, a central joint moiety such that each of the two or more linear peptides is connected to the central joint moiety via a linker, and a functionalized third linker connected to the central joint moiety. Preferably, the functionalized linker is conjugated to one or more radiolabeled moieties, such as one or more radiolabeled moieties disclosed herein. As illustrated in Scheme 3, in certain embodiments, each of the linkers can include a polyethylene glycol comprising 1 to about 2000 ethylene glycol units and having a molecular weight of about 200 to about 20,000, a sugar residue, or a short linker peptide. Preferably, the central joint moiety is an acidic amino acid moiety, for example, a glutamic acid moiety. Scheme 4 illustrates several exemplary radiofluorinated GPC3-binding dimer peptides for use with the invention.

Scheme 2. Illustrated is a schematic of one or more embodiments of a dimer construct for increased binding affinity to GPC3, resulting in improved pharmacokinetics of radiolabeled peptides.

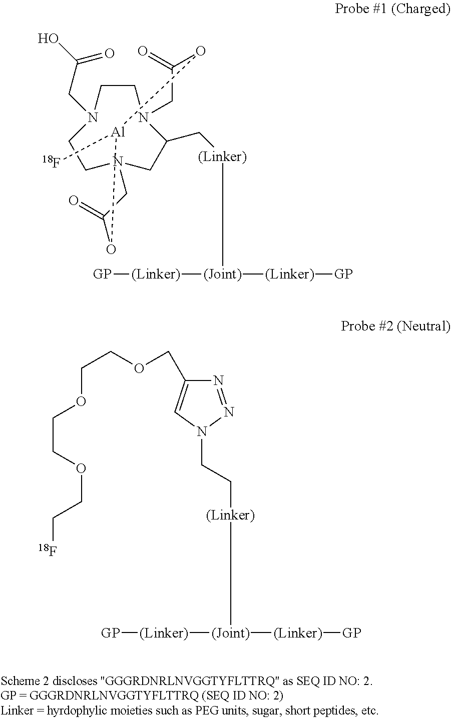

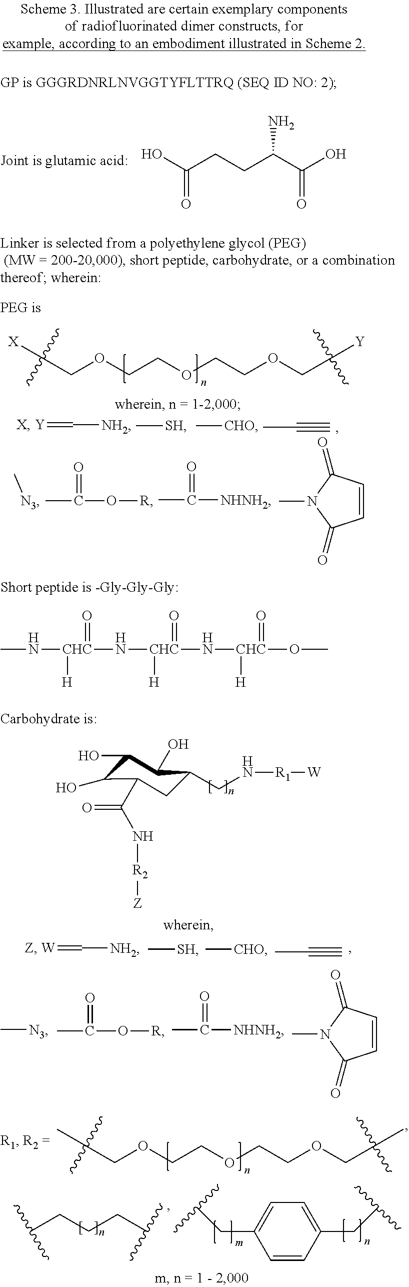

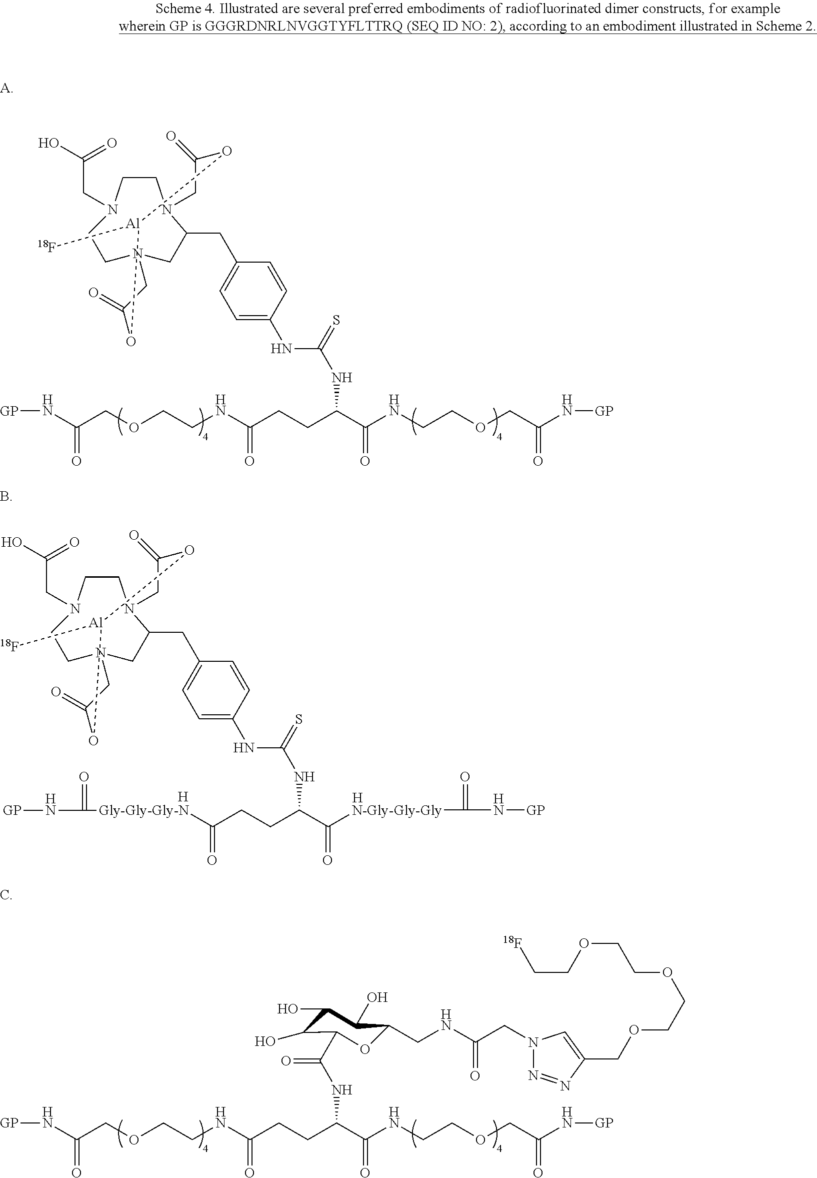

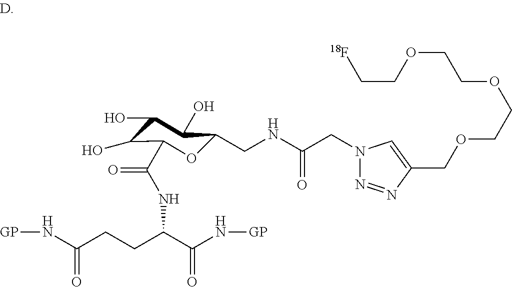

### Example 4

### Additional Embodiments of the Disclosed Technology

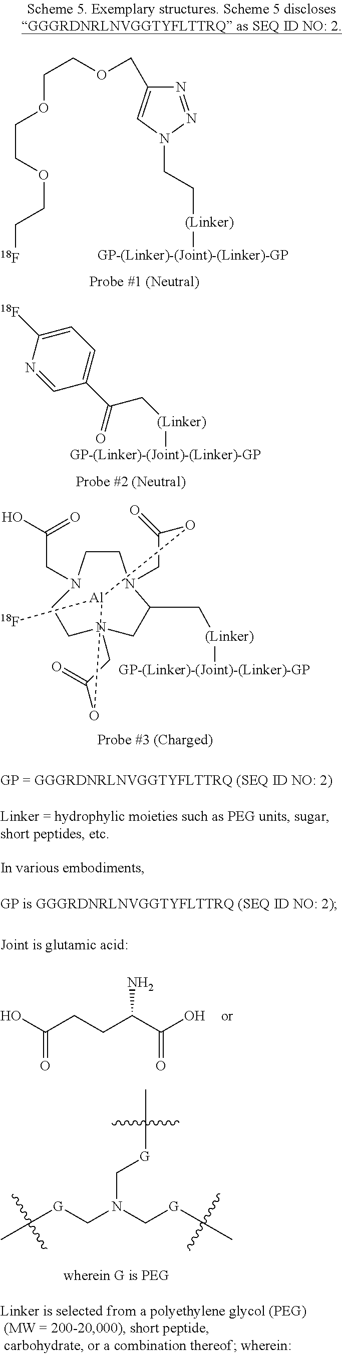

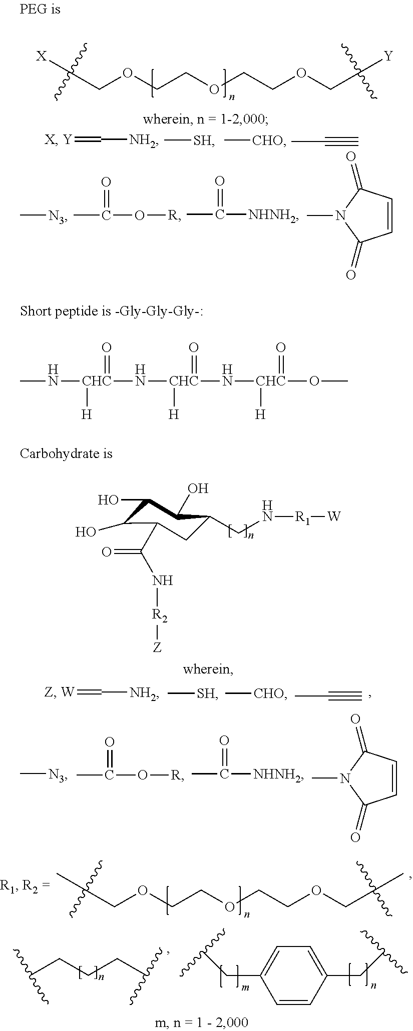

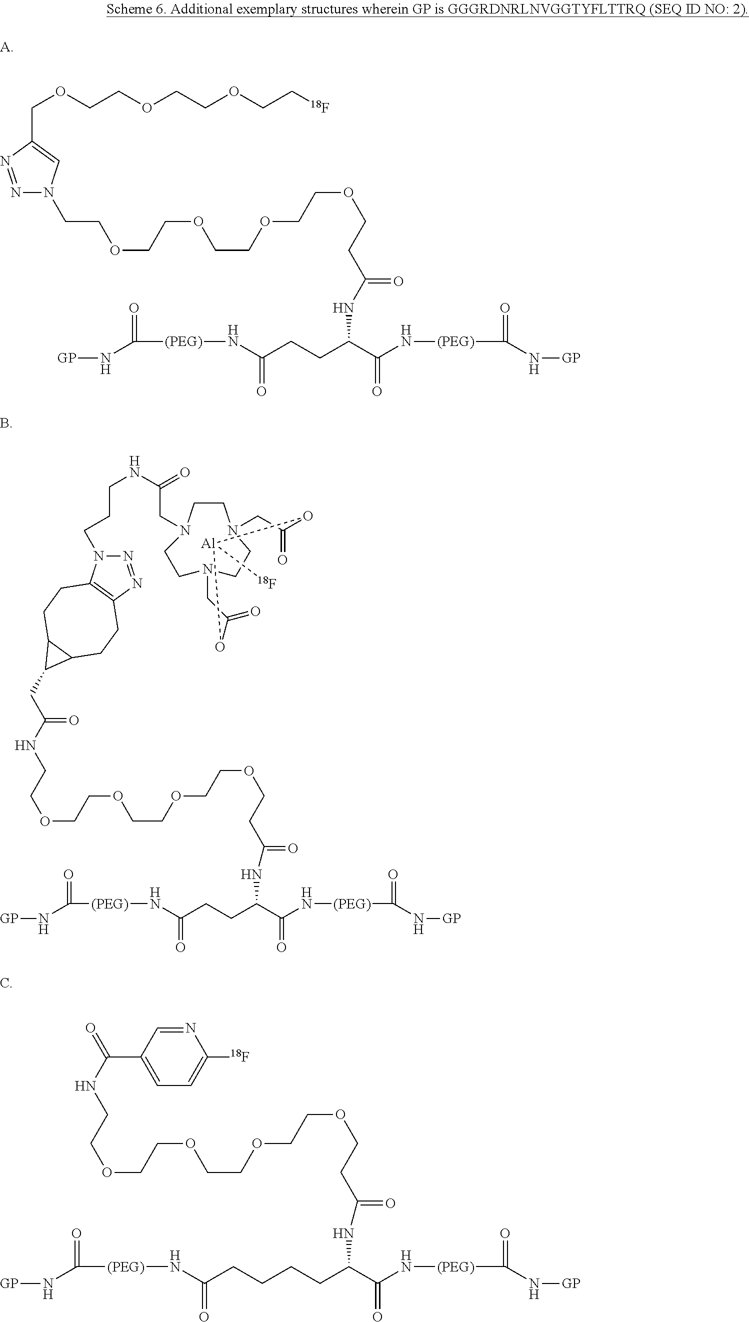

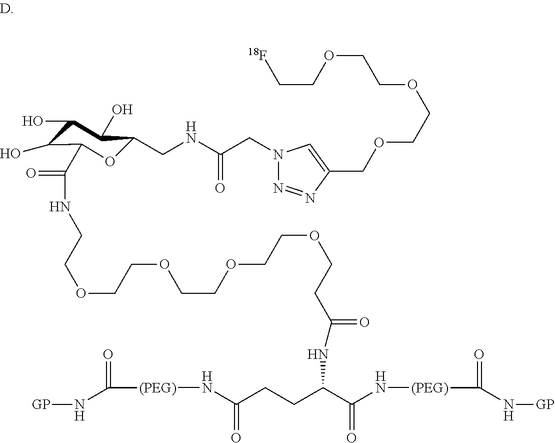

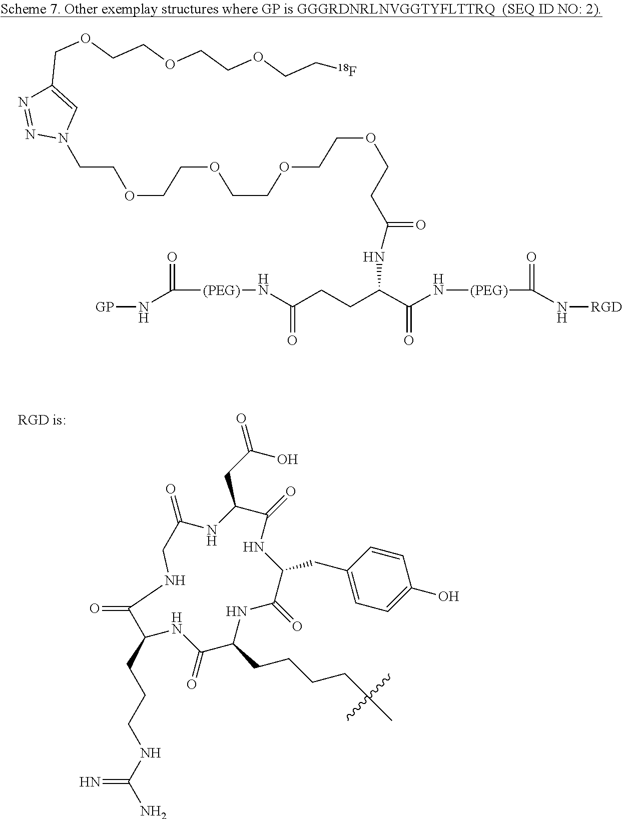

### Example 5

### Synthetic Scheme 8 and 9

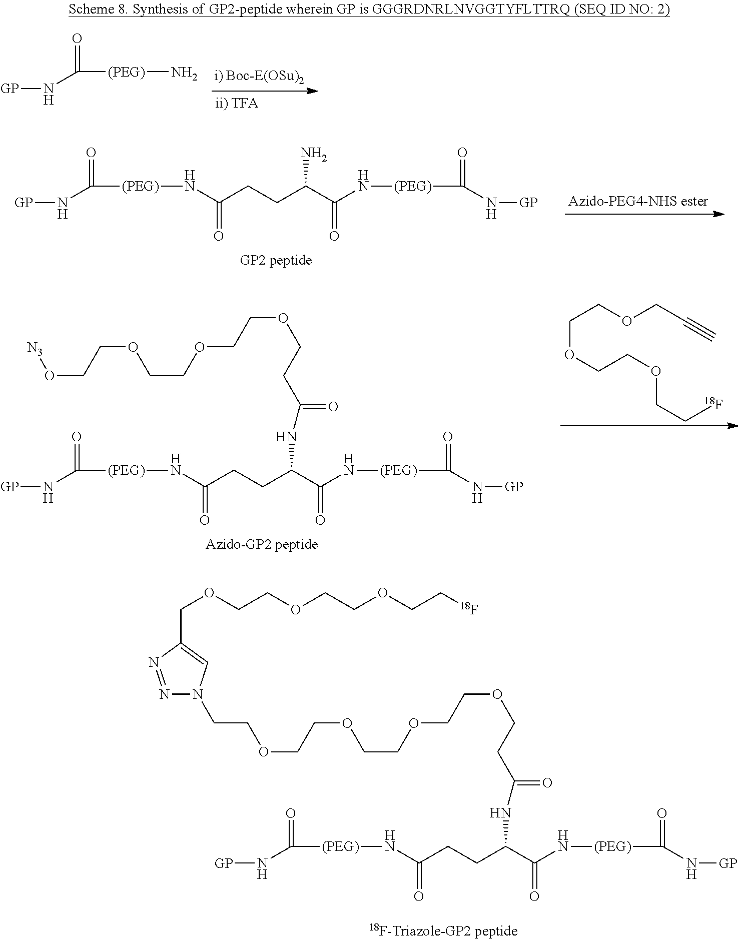

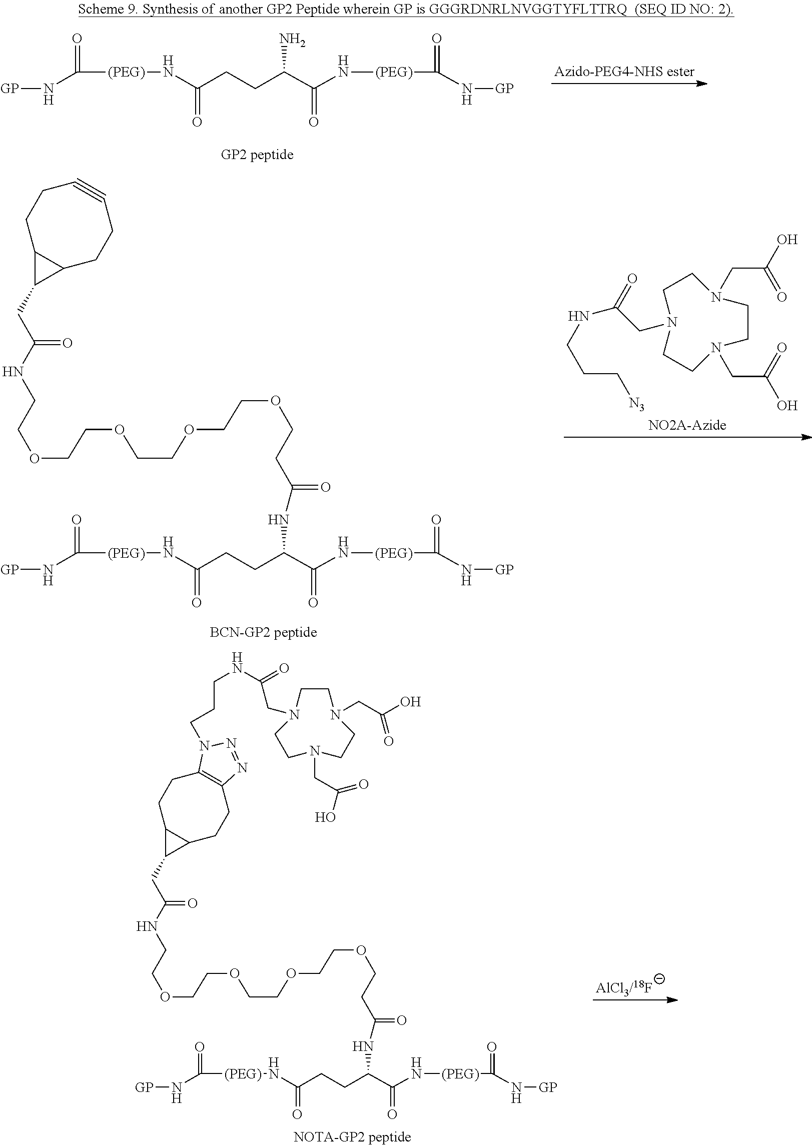

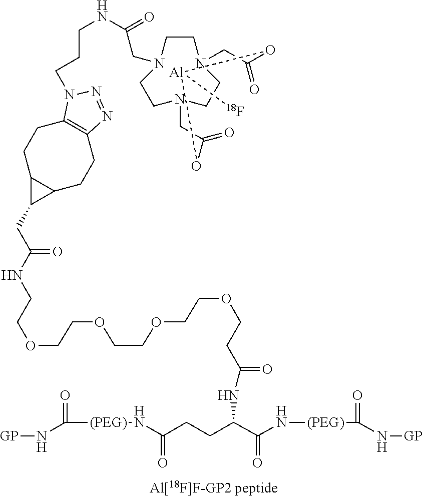

### Example 6

### Pharmaceutical Dosage Forms

The following formulations illustrate representative pharmaceutical dosage forms that may be used for the therapeutic or prophylactic administration of a composition described herein, or a composition specifically disclosed herein (hereinafter referred to as ‘Composition X’):

| (i) Tablet 1 | mg/tablet |
|---|---|
| ‘Composition X’ | 100.0 |
| Lactose | 77.5 |
| Povidone | 15.0 |
| Croscarmellose sodium | 12.0 |
| Microcrystalline cellulose | 92.5 |
| Magnesium stearate | 3.0 |
| | 300.0 |
| (ii) Tablet 2 | mg/tablet |
| ‘Composition X’ | 20.0 |
| Microcrystalline cellulose | 410.0 |
| Starch | 50.0 |
| Sodium starch glycolate | 15.0 |
| Magnesium stearate | 5.0 |
| | 500.0 |

| (iii) Capsule | mg/capsule |
|---|---|
| ‘Composition X’ | 10.0 |
| Colloidal silicon dioxide | 1.5 |
| Lactose | 465.5 |
| Pregelatinized starch | 120.0 |
| Magnesium stearate | 3.0 |
| | 600.0 |

| (iv) Injection 1 (1-75 mCi/mL) | mCi/mL |
|---|---|
| ‘Composition X’ | 1-75 mCi |
| Sodium chloride (0.9%) | q.s. |

| (v) Injection 2 (1-75 mCi/mL) | mCi/mL |
|---|---|
| ‘Composition X’ | 1-75 mCi |
| Dibasic sodium phosphate | q.s. |
| Monobasic sodium phosphate | q.s. |
| 1.0 N Sodium hydroxide solution | q.s. |
| (pH adjustment to 7.0-7.5) | |
| Water for injection | q.s. |
| (vi) Injection 3 (1 mg/mL) | mg/mL |
| ‘Composition X’ (free acid form) | 1.0 |
| Dibasic sodium phosphate | 12.0 |
| Monobasic sodium phosphate | 0.7 |
| Sodium chloride | 4.5 |
| 1.0 N Sodium hydroxide solution | q.s. |
| (pH adjustment to 7.0-7.5) | |
| Water for injection | q.s. ad 1 mL |
| (vii) Injection 4 (10 mg/mL) | mg/mL |
| ‘Composition X’ (free acid form) | 10.0 |
| Monobasic sodium phosphate | 0.3 |
| Dibasic sodium phosphate | 1.1 |
| Polyethylene glycol 400 | 200.0 |
| 0.1 N Sodium hydroxide solution | q.s. |
| (pH adjustment to 7.0-7.5) | |
| Water for injection | q.s. ad 1 mL |

| (viii) Aerosol | mg/can |
|---|---|
| ‘Composition X’ | 20 |
| Oleic acid | 10 |
| Trichloromonofluoromethane | 5,000 |
| Dichlorodifluoromethane | 10,000 |
| Dichlorotetrafluoroethane | 5,000 |

| (ix) Topical Gel 1 | wt. % |
|---|---|
| ‘Composition X’ | 5% |
| Carbomer 934 | 1.25% |
| Triethanolamine | q.s. |
| (pH adjustment to 5-7) | |
| Methyl paraben | 0.2% |
| Purified water | q.s. to 100 g |

| (x) Topical Gel 2 | wt. % |
|---|---|
| ‘Composition X’ | 5% |
| Methylcellulose | 2% |
| Methyl paraben | 0.2% |
| Propyl paraben | 0.02% |
| Purified water | q.s. to 100 g |

| (xi) Topical Ointment | wt. % |
|---|---|
| ‘Composition X’ | 5% |
| Propylene glycol | 1% |
| Anhydrous ointment base | 40% |
| Polysorbate 80 | 2% |
| Methyl paraben | 0.2% |
| Purified water | q.s. to 100 g |

| (xii) Topical Cream 1 | wt. % |
|---|---|
| ‘Composition X’ | 5% |
| White bees wax | 10% |
| Liquid paraffin | 30% |
| Benzyl alcohol | 5% |
| Purified water | q.s. to 100 g |

| (xiii) Topical Cream 2 | wt. % |
|---|---|
| ‘Composition X’ | 5% |
| Stearic acid | 10% |
| Glyceryl mono stearate | 3% |
| Polyoxyethylene stearyl ether | 3% |
| Sorbitol | 5% |
| Isopropyl palmitate | 2% |
| Methyl Paraben | 0.2% |
| Purified water | q.s. to 100 g |

These formulations may be prepared by conventional procedures well known in the pharmaceutical art. It will be appreciated that the above pharmaceutical compositions may be varied according to well-known pharmaceutical techniques to accommodate differing amounts and types of active ingredient ‘Composition X’. Aerosol formulation (vi) may be used in conjunction with a standard, metered dose aerosol dispenser. Additionally, the specific ingredients and proportions are for illustrative purposes. Ingredients may be exchanged for suitable equivalents and proportions may be varied, according to the desired properties of the dosage form of interest.

While specific embodiments have been described above with reference to the disclosed embodiments and examples, such embodiments are only illustrative and do not limit the scope of the invention. Changes and modifications can be made in accordance with ordinary skill in the art without departing from the invention in its broader aspects as defined in the following claims.

All publications, patents, and patent documents are incorporated by reference herein, as though individually incorporated by reference. No limitations inconsistent with this disclosure are to be understood therefrom. The invention has been described with reference to various specific and preferred embodiments and techniques. However, it should be understood that many variations and modifications may be made while remaining within the spirit and scope of the invention.

What is claimed is:

1. A binding probe of Formula I: 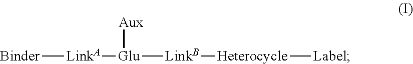
wherein
Label is Al[¹⁸F], ¹⁸F, Al[¹⁹F], ¹⁹F, or absent, wherein the bond to ¹⁸F or F is ionic or covalent;
Aux is absent or -Link-Binder;
Glu is absent or 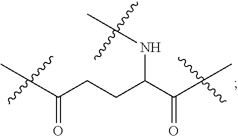 [SMILES: *NC(CCC(*)=O)C(*)=O]
Link^{A} is absent, a polyethylene glycol (PEG), or the short peptide GGG;
Link^{B} is 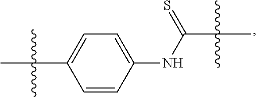 [SMILES: *C(=S)Nc1ccc(*)cc1]
a monosaccharide, PEG, or combination thereof;
Link^{C} is absent, a polyethylene glycol (PEG), or the short peptide GGG;
each Binder is independently a binding peptide (GP) or RGD;
wherein the binding peptide has a sequence identity of at least about 75% to the amino acid sequence -GGGRDNRLNVGGTYFLTTRQ (SEQ ID NO: 2) or -RLNVGGTYFLTTRQ (SEQ ID NO: 1);
wherein RGD is: 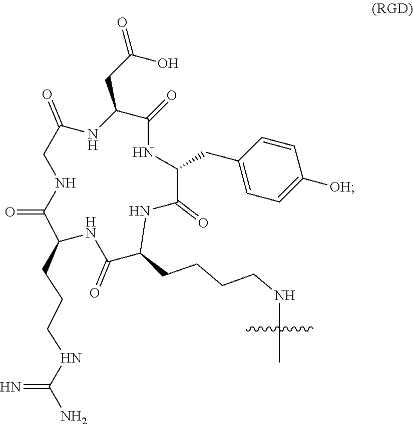 [SMILES: *NCCCC[C@@H]1NC(=O)[C@@H](Cc2ccc(O)cc2)NC(=O)[C@H](CC(=O)O)NC(=O)CNC(=O)[C@H](CCCNC(=N)N)NC1=O]
wherein the N-terminus of the binding peptide or the (CH₂)₄NH moiety of RGD is conjugated to Link^{B}, Link^{A}, or Glu, and the conjugate is via a thioacyl or acyl bond; and
Heterocycle is: 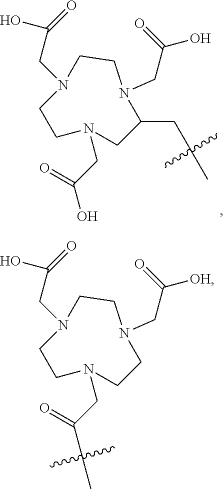
an imidazolyl, triazolyl, tetrazolyl, pyridyl, or combination thereof of any two of the heterocycles;
wherein Glu is not absent when Link^{A} and/or Link^{C} are not absent; and
each PEG independently has a molecular weight of about 20 kDa or less.

2. The probe of claim 1 wherein Link^{A} is conjugated via one or two amide bonds.

3. The probe of claim 1 wherein the heterocycle and/or monosaccharide and/or PEG is conjugated by one or more amide bonds.

4. The probe of claim 1 wherein the sequence identity is at least about 85%.

5. The probe of claim 1 wherein PEG is represented by Formula Ib: 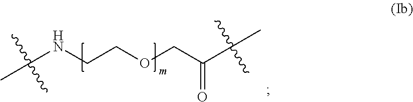
wherein m is 1-2000.

6. The probe of claim 1 wherein the monosaccharide is represented by Formula Ic: 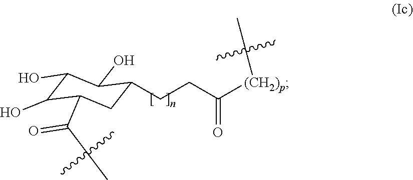
wherein n and p are each independently 0-2000.

7. The probe of claim 1 wherein the triazolyl is represented by Formula Id or Ie: 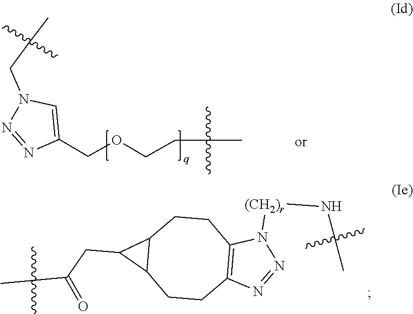
wherein q is 1-10; and r is 2-10.

8. The probe of claim 1 wherein the pyridyl is represented by Formula If:

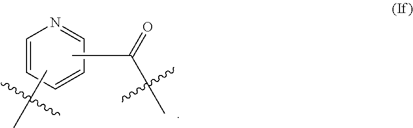

9. The probe of claim 1 wherein the Heterocycle is represented by Formula Ig: 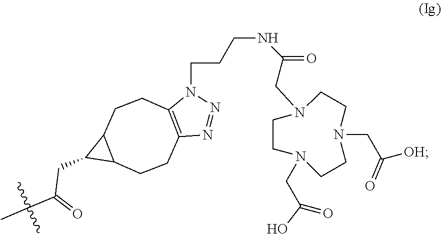
wherein s is 0-8.

10. The probe of claim 1 wherein the Label is Al[¹⁸F] or ¹⁸F.

11. The probe of claim 1 wherein both Aux and Glu are not absent.

12. The probe of claim 11 wherein both Link^{A} and Link^{C} are not absent.

13. The probe of claim 1 wherein the probe is represented by Formula II
Binder—Link^{B}—Heterocycle—Label (II);
wherein
Label is Al[¹⁸F] or ¹⁸F;
Binder is GP or RGD; and
Heterocycle is: 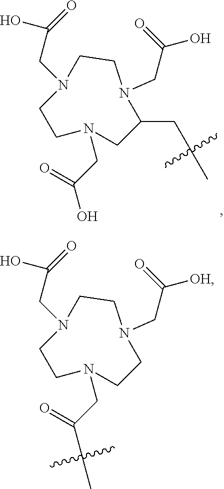
triazolyl, or pyridyl; wherein the sequence identity is at least about 95%.

14. The probe of claim 1 wherein the probe is: 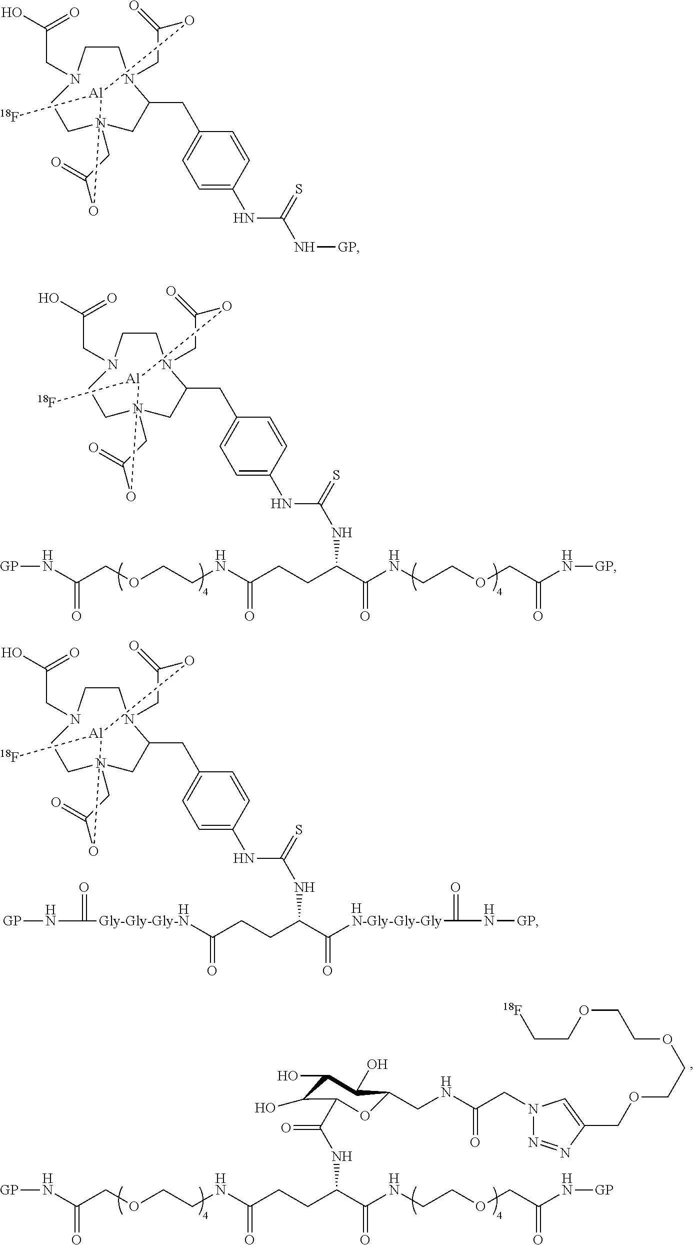 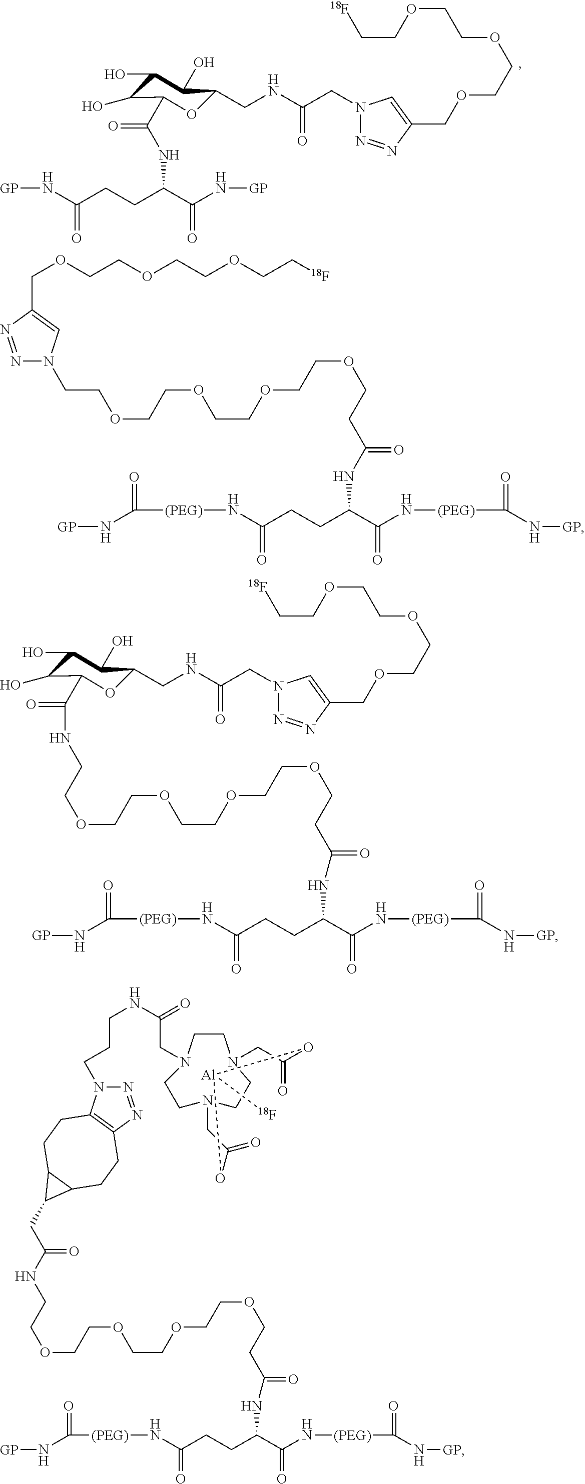 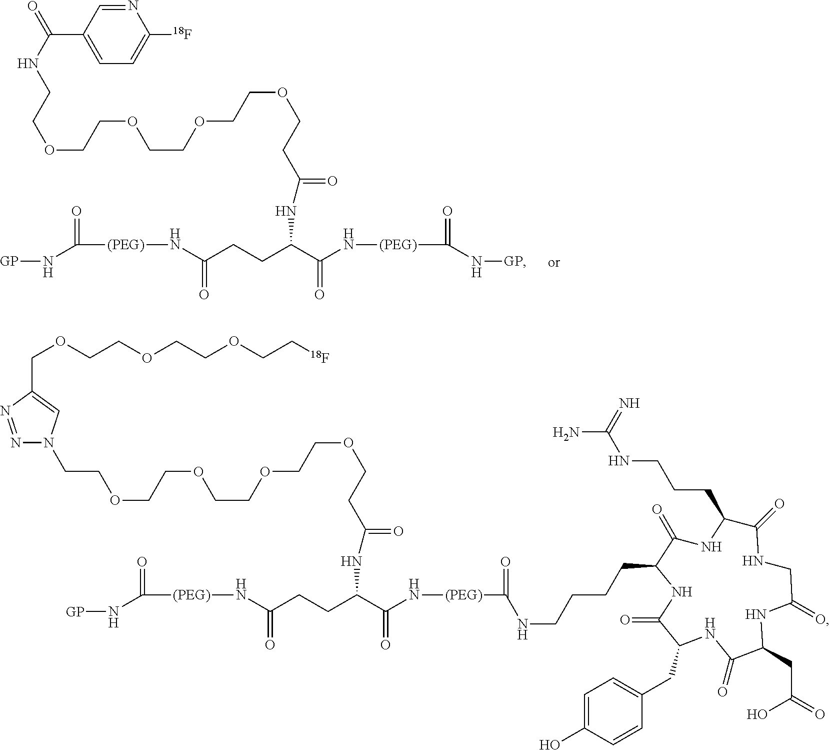
wherein
PEG consists of 4 repeat units; and
GP is -GGGRDNRLNVGGTYFLTTRQ (SEQ ID NO: 2) wherein the amine moiety of the amino acid G at the N-terminus of GP is represented as NH.

15. A method for imaging a cancer comprising:
a) administering an effective amount of the binding probe according to claim 1; and
b) imaging the presence or absence of the cancer in the subject.

16. The method of claim 15 wherein the probe is Al[¹⁸F]F-GP2633 or Al[¹⁸F]F-GP2076: 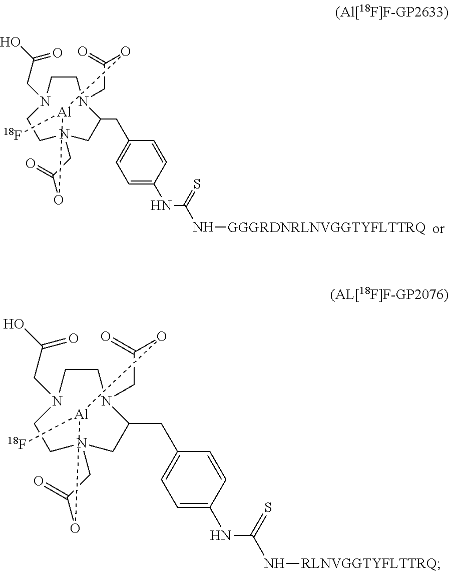
wherein the amine moiety of the amino acid G at the N-terminus of GGGRDNRLNVGGTYFLTTRQ (SEQ ID NO: 2) is represented as NH which forms the thiourea moiety in Al[¹⁸F]F-GP2633;
wherein the amine moiety of the amino acid R at the N-terminus of RLNVGGTYFLTTRQ (SEQ ID NO: 1) is represented as NH which forms the thiourea moiety in Al[¹⁸F]F-GP2026.

17. The method of claim 15 wherein the cancer is liver cancer or hepatocarcinoma.

18. A radiopharmaceutical composition comprising an ¹⁸F radiolabeled linear peptide that binds specifically to Glypican-3 (GPC3) expressed on a surface of a hepatocarcinoma cell; and
a pharmaceutically acceptable carrier.

19. The radiopharmaceutical composition of claim 18 wherein the radiolabeled linear peptide is at least 75% identical to amino acid sequence RLNVGGTYFLTTRQ (SEQ ID NO: 1) or GGGRDNRLNVGGTYFLTTRQ (SEQ ID NO: 2).

20. The radiopharmaceutical composition of claim 18 wherein the radiolabeled linear peptide is at least 95% identical to amino acid sequence RLNVGGTYFLTTRQ (SEQ ID NO: 1) or GGGRDNRLNVGGTYFLTTRQ (SEQ ID NO: 2).


SEQUENCE LISTING (3 sequences)

SEQ ID NO: 1 - protein, 14 aa, Artificial Sequence
  Description of Artificial Sequence Synthetic peptide
RLNVGGTYFLTTRQ

SEQ ID NO: 2 - protein, 20 aa, Artificial Sequence
  Description of Artificial Sequence Synthetic peptide
GGGRDNRLNVGGTYFLTTRQ

SEQ ID NO: 3 - protein, 6 aa, Artificial Sequence
  Description of Artificial Sequence Synthetic peptide
GGGRDN